SEQUENCE LISTING (2 sequences)

SEQ ID NO: 1 - protein, 467 aa, Homo sapiens [sequence omitted]

SEQ ID NO: 2 - DNA, 2763 bp, Homo sapiens
ggatcccagtggcccggcgtgctcggctcccacaggcctgcagccagcatcgcaccgaaccttcggggggccgcggctggagcgctcggccggcgtgggagcgcaaggccgcagatgcaatcttcttaccgcgaagaagccaggggaataggtagccacatcttgtttgcagataagaaaggaagctaacgcagtatctgcaaagccaggagtctgactcagtacttttctcactcatgcatacaagcagctaaaaatgacacagcttatttaccatgcccctgacactgcactgagcactttatgagcttgaactctgttaatctcacgaccacctcatgagactctccagaaagagcaacagtaatggagtacatgagcactggaagtgacaataaagaagagattgatttattaattaaacatttaaatgtgtctgatgtaatagacattatggaaaatctttatgcaagtgaagagccagcagtttatgaacccagtctaatgaccatgtgtcaagacagtaatcaaaacgatgagcgttctaagtctctgctgcttagtggccaagaggtaccatggttgtcatcagtcagatatggaactgtggaggatttgcttgcttttgcaaaccatatatccaacactgcaaagcatttttatggacaacgaccacaggaatctggaattttattaaacatggtcatcactccccaaaatggacgttaccaaatagattccgatgttctcctgatcccctggaagctgacttacaggaatattggttctgattttattcctcggggcgcctttggaaaggtatacttggctcaagatataaagacgaagaaaagaatggcgtgtaaactgatcccagtagatcaatttaagccatctgatgtggaaattcaggcttgcttccggcacgagaacatcgcagagctgtatggcgcagtcctgtggggtgaaactgtccatctctttatggaagcaggcgagggagggtctgttctggagaaactggagagctgtggaccaatgagagaatttgaaattatttgggtgacaaagcatgttctcaagggacttgattttctacactcaaagaaagtgatccatcatgatattaaacctagcaacattgttttcatgtccacaaaagctgttttggtggattttggcctaagtgttcaaatgaccgaagatgtctattttcctaaggacctccgaggaacagagatttacatgagcccagaggtcatcctgtgcaggggccattcaaccaaagcagacatctacagcctgggggccacgctcatccacatgcagacgggcaccccaccctgggtgaagcgctaccctcgctcagcctatccctcctacctgtacataatccacaagcaagcacctccactggaagacattgcagatgactgcagtccagggatgagagagctgatagaagcttccctggagagaaaccccaatcaccgcccaagagccgcagacctactaaaacatgaggccctgaacccgcccagagaggatcagccacgctgtacgagtctggactctgccctcttggagcgcaagaggctgctgagtaggaaggagctggaacttcctgagaacattgctgattcttcgtgcacaggaagcaccgaggaatctgagatgctcaagaggcaacgctctctctacatcgacctcggcgctctggctggctacttcaatcttgttcggggaccaccaacgcttgaatatggctgaaggatgccatgtttgcctctaaattaagacagcattgatctcctggaggctggttctgctgcctctacacaggggcccgttacagtgaatggtgccattttcgaaggagcagtgtgacctcctgtgacccatgaatgtgcctccaagcggccctgtgtgtttgacatgtgaagctatttgatatgcaccaggtctcaaggttctcatttctcaggtgacgtgattctaaggcaggaatttgagagttcacagaaggatcgtgtctgctgactgtttcattcactgtgcactttgctcaaaattttaaaaataccaatcacaaggataatagagtagcctaaaattactattcttggttcttatttaagtatggaatattcattttactcagaatagcctgttttgtgtatattggtgtatattatataactctttgagcctttattggtaaattctggtatacattgaattcattataatttgggtgactagaacaacttgaagattgtagcaataagctggactagtgtcctaaaaatggctaactgatgaattagaagccatctgacagacggccactagtgacagtttcttttgtgttcctatggaaacattttatactgtacatgctatgctgaagacattcaaaacgtgatgttttgaatgtggataaaactgtgtaaaccacataattttgtacatccaaggatgaggtgtgacctttaagaaaaatgaaaacttttgtaaattattgatgattttgtaattcttatgactaaattttcttttaagcatttgtatattaaaatagcatactgtgtatgttttatatcaaatgccttcatgaatctttcatacatatatatatttgtaacatgtaaagtatgtgagtagtcttatgtaaagtatgtttttacattatgcaaataaaacccaatacttttgtccaatgtggttggtcaaatcaactgaataaattcagtattttgcctt

(19) United States
(12) Patent Application Publication
(10) Pub. No.: US 2022/0313700 A1
(43) Pub. Date: Oct. 6, 2022

(54) METHODS FOR TREATING MAP3K8 POSITIVE CANCERS

(71) Applicant: CELL RESPONSE, INC., San Bruno, CA (US)

(72) Inventor: Yoram ALTSCHULER, San Bruno, CA (US)

(21) Appl. No.: 17/433,818

(22) PCT Filed: Feb. 26, 2020
(86) PCT No.: PCT/US2020/020004

§ 371 Date: Aug. 25, 2021

Related U.S. Application Data
(60) Provisional application No. 62/810,520, filed on Feb. 26, 2019.

(51) Int. Cl. A61K 31/5377; A61K 31/506; A61K 31/517; A61K 31/4545; A61K 39/395; A61K 31/675; A61K 31/439; A61K 31/519; A61K 31/454; A61K 31/502; A61K 31/55; A61K 31/5025; A61K 31/4709; A61K 31/192; A61K 31/496; A61K 31/5513; A61K 31/4184; A61K 31/44; A61K 31/52; A61K 31/4439; A61K 31/436; A61K 31/4706; A61K 31/47; A61P 35/00

(52) U.S. Cl. CPC A61K 31/5377; A61K 31/506; A61K 31/517; A61K 31/4545; A61K 39/3955; A61K 31/675; A61K 31/439; A61K 31/519; A61K 31/454; A61K 31/502; A61K 31/55; A61K 31/5025; A61K 31/4709; A61K 31/192; A61K 31/496; A61K 31/5513; A61K 31/4184; A61K 31/44; A61K 31/52; A61K 31/4439; A61K 31/436; A61K 31/4706; A61K 31/47; A61P 35/00

(57) ABSTRACT

This invention provides for a method of improving chemotherapeutic, targeted therapy or immunotherapeutic treatments for cancers arising from oncogene activity. The method provides for the combination of a COT inhibitor with a therapeutic specific for the oncogene. Methods of diagnosing the status of the patients, who are predisposed to benefit from this combination therapy are also described here. The use of any COT inhibitor in combination with other drugs for the treatment of cancer patients expressing COT. COT expression serves as a way for several oncogenes to stimulate proliferation or anti-apoptotic signaling that compromises drugs clinical benefit. To avoid that reduced clinical benefit, COT inhibitors may be combined with one or two other drugs that together will provide additional clinical benefit. The drugs to be combined can be part of a list of targeted therapy drugs that target: (i) growth factor inhibitors and growth factor receptor inhibitors; (ii) Fusion proto-oncogene inhibitors; (iii) proto-oncogene GTPases of 19 to 23 kDa and associated proteins inhibitors: (iv) proto-oncogenic cytoplasmic tyrosine and serine/threonine kinases inhibitors: (v) multi-kinase inhibitors: and (vi) cell cycle or DNA repair inhibitors.

### CROSS-REFERENCES TO RELATED APPLICATIONS

The present application claims priority to U.S. Provisional Patent Application No. 62/810,520, filed on Feb. 26, 2019, which application is incorporated herein by reference in its entirety.

### STATEMENT AS TO RIGHTS TO INVENTIONS MADE UNDER FEDERALLY SPONSORED RESEARCH AND DEVELOPMENT

Not Applicable

### SEQUENCE LISTING

The Sequence Listing submitted electronically is a text file of 8 KB, named ‘CRI_001_SQL’, and created Feb. 28, 2022, hereby incorporated by reference.

### FIELD OF THE INVENTION

This invention provides for diagnostic and therapeutic treatments of MAP3K8 positive cancers together with drugs that target non-MAP3K8 oncogenes.

### BACKGROUND OF THE INVENTION

It is known that many cancers arise from either cancer suppression genes being downregulated or from oncogenes that are either upregulated or mutated to allow for uncontrolled cell proliferation, or both. Treatment of cancers arising from oncogenes often involve administration of targeted inhibitors that interfere with the cell signalling that has gone awry. Cells representing oncogene-based cancers are known to respond to targeted inhibitors and subsequently becoming resistant to the inhibitor or to cells that express a gene that activates a signaling pathway that compromises drug treatment clinical benefit (Innate resistance). The problem being solved by this invention relates to overcoming resistance by co-administration of a MAP3K8 inhibitor with a targeted oncogene inhibitor.

### BRIEF SUMMARY OF THE INVENTION

This invention provides a method of treating a patient suffering from a MAP3K8 (COT) positive cancer. The method comprises the administration of an effective amount of a combination therapy comprising a COT inhibitor and at least one therapeutic agent selected from the group consisting of: (i) growth factor inhibitors and growth factor receptor inhibitors; (ii) Fusion proto-oncogene inhibitors; (iii) proto-oncogene GTPases of 19 to 23 kDa and associated proteins inhibitors; (iv) proto-oncogenic cytoplasmic tyrosine and serine/threonine kinases inhibitors; (v) multi-kinase inhibitors and (vi) cell cycle or DNA repair inhibitors.

Preferred cancer therapeutics include growth factor inhibitors and growth factor receptor inhibitors. Preferred cancer therapeutic drugs include, but are not limited to, Osimertinib, Afatinib, Panitumumab, Cetuximab, Trastuzumab, Crizotinib, and Imatinib.

When the preferred cancer therapeutic is a fusion proto-oncogene inhibitor, preferred drugs include Alectinib, Crizotinib, Ceritinib, Brigatinib or Lorlatinib.

When the preferred cancer therapeutic is a cell cycle or DNA repair inhibitor, preferred drugs are Abemaciclib, Trilaciclib, niraparib, olaparib, rucaparib, or talazoparib.

When the preferred cancer therapeutic is a proto-oncogene GTPase of 19 to 23 kDa and associated proteins, preferred drugs include ARS-853; ARS-1620; AMG-510 (CAS number 2252403-56-6, which is also known as 6-fluoro-7-(2-fluoro-6-hydroxyphenyl)-1-[4-methyl-2-(propan-2-yl)pyridin-3-yl]-4-[(2S)-2-methyl-4-(prop-2-enoyl)piperazin-1-yl]-1H,2H-pyrido[2,3-d]pyrimidin-2-one; MTRX849 (HRAS, NRAS and KRAS inhibitors), tipifarnib, lonafarnib, bms-214662, 1778123 (farnesyl transferase inhibitors), deltarasin (KRAS-PDEδ inhibitors), sulindac-derived compounds (Ras-Raf interaction inhibitors), and Kobe0065 or Kobe2602 (SOS binding inhibitors).

When the preferred the cancer therapeutic is a protooncogenic cytoplasmic tyrosine kinase, a serine/threonine kinase or a membrane lipid kinase, preferred drugs include (i) Trametinib, Binimetinib, and Sorafenib (MEK1/2 inhibitors), (ii) SCH772984, GDC-0994, Ulixertinib, and LY3214996 (ERK1/2 inhibitors), (iii) Duvelisib, Copanlisib, and Copanlisib (PI3K inhibitors), (iv) Everolimus, Sirolimus and Temsirolimus (mTOR inhibitors), or (v) idelalisib (AKT inhibitors).

When the preferred cancer therapeutic is a multi-kinase inhibitor, preferred drugs include neratinib, ponatinib, regorafenib, sorafenib, cabozantinib, lenvatinib, or vandetanib.

The above methods, including the preferred drug classes and drugs, find use when the MAP3K8 (COT) positive cancer is selected from the group consisting of: pancreatic cancer, renal cancer, breast cancer, bladder cancer, leukemia, acute myeloid leukemia, thyroid cancer, colorectal cancer, prostate cancer, uterine carcinosarcoma, uterine cancer, bladder urothelial carcinoma, uterine corpus endometrial carcinoma, gastric adenocarcinoma, cervical adenocarcinoma, hepatocellular cancer, lung cancer (NSCLC, SCLC, lung adenocarcinoma, lung squamous cell carcinoma), glioblastoma multiforme, glioblastoma, brain cancer, ovarian cancer, cervical cancer, gastric cancer, esophageal cancer, head and neck cancer, melanoma, skin cancer, neuroendocrine cancers, multiple myeloma, brain tumors (e.g., adult glioblastoma multiforme; glioma, anaplastic oligodendroglioma, and adult anaplastic astrocytoma), child brain tumor, bone cancer, sarcoma, CNS cancer, ovarian cancer, renal cancer, prostate cancer, or breast cancer.

In addition to methods of treatment, this invention provides a method of evaluating a patient with a cancer selected from the group including but not limited to the following: where the cancer has an oncogene status of either elevated expression levels of an oncogene or harboring an oncogenic mutation where the oncogene is selected from the group consisting of the following: (i) growth factor and growth factor receptor oncogene; (ii) Fusion of proto-oncogenes; (iii) GTPases oncogene of 19 to 23 Kda and/or associated proteins as supporting oncogenic activity; and, (iv) oncogenic cytoplasmic tyrosine and serine/threonine kinases. The method of evaluating a patient with cancer comprises the following steps: (i) determining the MAP3K8 (COT) status in patient tumor cells from a patient sample; (ii) comparing the levels of COT expression or activation (by phosphorylation) from step (a) to a threshold activity level of COT derived from a cohort of cells from at least 200 test individuals, where the cells have a defined level of COT activation/expression that is either negative expression or positive expression where the cohort of cells represent a cancer having a positive oncogene status; (iii) determining the oncogene status of the cancer cells from the patient; and, (iv) identifying the patient as potentially responding therapeutically to a combination of a COT inhibitor and an oncogene inhibitor that is known to therapeutically treat cancers matching the oncogene status of step (iii). Preferred embodiments are the same as those set forth above for the methods of treatment where preferred therapy combinations are described. Other preferred methods for determining cancer status are described in this application including where the COT status is determined by amplification of mRNA encoding COT or the phosphorylation of COT, or copy number of COT, or mutation of COT or COT overexpression (elevated protein level). Alternative embodiments include those where the COT status is determined on patient tumor (biopsy or liquid biopsy) utilizing a method selected from the group including, but not limited to, polymerase chain reaction, isothermal amplification (PCR), Immuno-histochemistry with or without anti-phopho antibodies to Thr290 of COT, Next Generation Sequencing, liquid biopsy, and direct biopsy.

This invention further provides for a method of treating a patient hosting a MAP3K8 positive cancer where the method involves first selecting a patient determined to have an oncogenic originating cancer and an elevated COT status. The method comprising: selecting a patient with a cancer selected from the group consisting of: where the cancer has an oncogene status of either elevated expression levels of an oncogene or harboring an oncogenic mutation, where the oncogene is selected from the group consisting: (i) growth factor and growth factor receptor oncogene; (ii) Fusion of proto-oncogenes; (iii) GTPases oncogene of 19 to 23 Kda and/or associated proteins as supporting oncogenic activity; and, (iv) oncogenic cytoplasmic tyrosine and serine/threonine kinases; where the patient is also determined to have cancer cells having an elevated MAP3K8 (COT) status, where that elevated COT status is determined by comparing the levels of COT expression or activation (by phosphorylation) to a threshold activity level of COT derived from a cohort of cells from at least 200 test individuals, where the cells have a defined level of COT activation/expression that is either negative expression or positive expression, where the cohort of cells represent a cancer having a positive oncogene status and, then treating the patient with a therapeutically effective amount of a combination of a COT inhibitor and an oncogene inhibitor that is known to therapeutically treat cancers matching the oncogene status of the patient. All of the above recited embodiments apply to this method.

### BRIEF DESCRIPTION OF THE DRAWINGS

FIG. 1: Quinoline based COT inhibitors.

FIG. 2: Indazoles based COT inhibitors.

FIG. 3: Thieno based COT inhibitors.

FIG. 4: COT inhibitor used in examples below.

FIG. 5: Proliferation of NCSLC cell lines harboring oncogenic KRAS are inhibited by combination therapy of COT inhibitor with MEK inhibitor Trametinib.

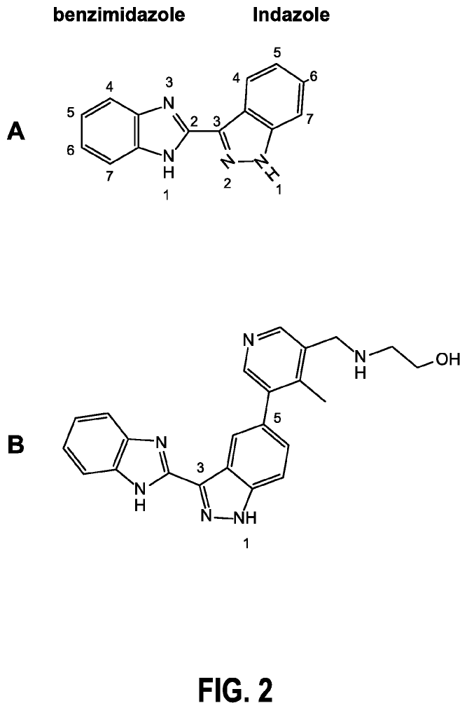

FIG. 2: Indazoles based COT inhibitors.

FIG. 6: Proliferation of NCSLC cell line A549 harboring oncogenic KRAS are inhibited by combination therapy of COT inhibitor with ERK inhibitor Ulixertinib.

FIG. 7: Proliferation of colorectal cancer cell lines harboring oncogenic KRAS are inhibited by combination therapy of COT inhibitor with MEK inhibitor Trametinib.

FIG. 8: Proliferation of colorectal cancer cell lines harboring oncogenic KRAS are inhibited by combination therapy of COT inhibitor with ERK inhibitor Ulixertinib.

FIG. 9: Proliferation of colorectal cancer cell lines harboring oncogenic KRAS are inhibited by combination therapy of COT inhibitor with PI3K/mTOR inhibitor Gedatolisib.

FIG. 10: Proliferation of colorectal cancer cell lines harboring oncogenic KRAS are inhibited by combination therapy of COT inhibitor with mTOR inhibitor Everolimus.

FIG. 11: Proliferation of pancreatic cancer cell lines harboring oncogenic mutations are inhibited by combination therapy of COT inhibitor with MEK inhibitor Trametinib.

FIG. 12: Proliferation of pancreatic cancer cell lines harboring oncogenic mutations are inhibited by combination therapy of COT inhibitor with ERK inhibitor Ulixertinib.

FIG. 13: Proliferation of colorectal cancer cell line H29 harboring oncogenic BRAF are inhibited by combination therapy of COT inhibitor with either MEK inhibitor trametinib or PI3K inhibitor Gedatolisib.

FIG. 14: Proliferation of NSCLC cancer cell lines harboring oncogenic EGFR are inhibited by combination therapy of COT inhibitor with EGFR inhibitor Afatinib.

FIG. 15: Proliferation of NSCLC cancer cell lines harboring oncogenic EGFR are inhibited by combination therapy of COT inhibitor with EGFR inhibitor Osimertinib.

FIG. 16: Proliferation of NSCLC cancer cell lines harboring oncogenic EGFR are inhibited by combination therapy of COT inhibitor with MEK inhibitor Trametinib.

FIG. 17: Proliferation of NSCLC cancer cell lines harboring oncogenic EGFR are inhibited by combination therapy of COT inhibitor with VEGFR inhibitor vandetanib.

FIG. 18: Proliferation of NSCLC cancer cell line STE1 harboring oncogenic EML4-ALK is inhibited by combination therapy of COT inhibitor with Lorlatinib.

FIG. 19: Proliferation of colorectal cancer cell line HCT15 harboring oncogenic KRAS and PI3K are inhibited by triple combination therapy of COT inhibitor with PI3K inhibitor and MEK inhibitor.

FIG. 20: Proliferation of colorectal cancer cell lines harboring oncogenic KRAS and PI3K are inhibited by triple combination therapy of COT inhibitor with PI3K inhibitor and ERK inhibitor.

FIG. 21: Proliferation of NSCLC cancer cell line H1975 harboring oncogenic EGFR is inhibited by KD of either EGFR or COT or by combination of EGFR inhibitor with KD of COT.

FIG. 22: Proliferation of NSCLC cancer cell lines harboring oncogenic KRAS or NF1^{LOF} is mainly inhibited by KD of COT.

FIG. 23: Proliferation of colorectal cancer cell lines harboring oncogenic KRAS and oncogenic PI3K is inhibited mainly by KD of COT.

FIG. 24: Proliferation of pancreatic cancer cell line harboring diverse oncogenes is inhibited by KD of either BRAF, COT or EGFR.

### ABBREVIATIONS USED IN THE APPLICATION

Mitogen Activated Protein Kinase—MAPK

Mitogen Activated Protein Kinase Kinase Kinase 8—MAP3K8

Cancer Osaka Thyroid—COT

Tumor Progression Locus 2—TPL2

Trametinib—Tram.

Omipalisib—Omipa.

Everolimus—Ever.

Gedatolisib—Geda.

Vandetanib—Vandet.

Mitogen Activated Protein Kinase—MAPK

Epidermal Growth Factor Receptor—EGFR

Phosphatidylinositol 3-kinase—PI3K

Vascular Endothelial Growth Factor Receptor—VEGFR

Anaplastic Lymphoma Kinase—ALK

Growth Factor (GF)

Growth Factor Receptor” (GFR)

Wild Type—WT

Metastatic ColoRectal Cancer—mCRC

Non-Small Cell Lung Carcinoma—NSCLC

Receptor Tyrosine Kinases (RTK)

Extracellular signal-regulated kinases (ERK)

Overall survival (OS)

Objective Response Rate (ORR) or Response rate (RR)

Complete response (CR)

Duration of response (DoR)

Pathological complete response: pCR

Immune-related response criteria (iRECIST)

Minimal residual disease: MRD

Progression-free survival (PFS)

Horseradish peroxidase—HRP)

Chromogenic In Situ Hybridization—CISH

Fluorescence In-Situ Hybridization—FISH

Formalin-Fixed, Paraffin-Embedded—FFPE

Polymerase Chain Reaction—PCR

Next-Generation Sequencing—NGS

Reverse transcription polymerase chain reaction—RTPCR

Quantitative polymerase chain reaction—qPCR

Knockdown—KD

Knockout—KO

Inhibitor—In.

### DETAILED DESCRIPTION OF THE INVENTION

### Introduction

Cancer arises, at least in part, from the uncontrolled and inappropriate proliferation of malignant cells in the body. A major cause of this abnormal proliferation is due to aberrations in the signaling pathways that control the proliferation of healthy cells in the body. Abnormal signaling in cancer has many causes. Two of the broad categories of such causes include mutations or changes in copy number, or both, of genes encoding proteins that are involved in these signaling pathways. Genes that have been mutated or changed in copy number so as to promote cancerous behavior of cells, or cause cancer in human patients or laboratory animals, or any combination of these, are often referred to as “oncogenes.” The abnormal proliferation found in cancer, oncogenes, the abnormal signaling pathways found in cancers, and their causes are all described in, e.g., The Biology of Cancer, 2^{nd} Edition, Robert A. Weinberg, W. W. Norton and Co., 2013.

In recent years, several types of inhibitors have been developed that when added to cancerous cells in culture, or when administered to patients suffering from cancer, can counteract or block the effect of specific oncogenes and can thus be used to treat certain cancers. These types of inhibitors are often referred to as “targeted therapies.” The use of targeted inhibitors aims to block the abnormal signaling supporting abnormally increased proliferation arising from an oncogene. For example, the Epidermal Growth Factor Receptor (EGFR) normally controls the proliferation of many cell types in the body. EGFR can transmit a proliferative signal through a cascade of protein kinases termed the Mitogen Activated Protein Kinase (MAPK) pathway. Within the same cell, additional oncogenes can transmit additional proliferating signals. The EGFR is frequently mutated in certain types of cancer, such as Non-Small Cell Lung Carcinoma (NSCLC). Once a cancer therapeutic treatment is initiated by administration of a drug that inhibits the mutant EGFR oncogenic signaling, most of the cells that previously were proliferating will instead die by apoptosis, thereby shrinking the tumor. However, one or more cells within the tumor can harbor a resistance mechanism to the drug treatment. This resistance mechanism is often based on a mutated gene, whose expression enables the expression of a proliferation signal in the cell to continue despite the drug treatment. When this happens, these cells continue to proliferate despite the presence of the drug treatment causing the relapse of the disease.

A cancer-related resistance gene has now been identified whose protein product is known by several names, including Mitogen Activated Protein Kinase Kinase Kinase 8 (MAP3K8), Cancer Osaka Thyroid (COT), and Tumor Progression Locus 2 (TPL2). The multiple names reflect that this gene and the corresponding protein have been discovered multiple times. However, the present invention is concerned with the human gene and protein, though work in the field has also concentrated on the murine ortholog, as well as in other species. The various names and abbreviations are used herein interchangeably. COT is a serine/threonine kinase, and, it is related to other members of the MAPK family by structure and sequence homology.

COT is widely expressed in cancerous cells of malignant tumors, where COT transmits a proliferative signal and causes one or more cancerous cells of a malignant tumor to be resistant to drugs or other therapies used to treat the malignant tumor. Administration of a specific inhibitor of COT combined with the administration of an appropriate inhibitor of another oncogenic gene, or oncogenic protein, or signaling pathway, or other agent or mechanism that can contribute to the malignant behavior (termed here an “additional therapeutic”), can inhibit cancerous cell growth, or promote cancerous cell death, or both.

This invention provides for a method of improving chemotherapeutic or immunotherapeutic treatments of cancers arising from oncogene activity. The method provides for the combination of a COT inhibitor with a therapeutic specific for the oncogene or other drug target whose inhibition provides an anti-cancer effect (for example, CDK4/6 or PARP inhibitors). Methods of diagnosing the status of the patients, who are predisposed to benefit from this combination therapy, are also described here.

### Definitions

Unless defined otherwise, all technical and scientific terms used herein have the same meaning as is commonly understood by one of skill in the art to which the invention(s) belong. All patents, patent applications, published applications and publications, Genbank sequences, Pubmed ID, Pubmed Central, Protein Data Bank, Entrez, UniProt, HUGO, OMIM, websites and other published materials referred to throughout the entire disclosure herein, unless noted otherwise, are incorporated by reference in their entirety. Where reference is made to a URL or other such identifier or address, it understood that such identifiers can change and particular information on the internet can come and go, but equivalent information can be found by searching the internet. Reference thereto evidences the availability and public dissemination of such information.

The word “oncogene” refers to a gene that has the potential to cause cancer. In tumor cells, they are often mutated and/or expressed at abnormally high levels. Most normal cells will undergo a programmed form of rapid cell death (apoptosis) when critical functions are altered and malfunctioning. Activated oncogenes can cause those cells designated for apoptosis to survive and proliferate instead. Most oncogenes began as proto-oncogenes, normal genes involved in cell growth and proliferation or inhibition of apoptosis. If normal genes promoting cellular growth, through mutation, are activated through a gain of function mutation, other form of mutation, or increased in activity through other means, such as overexpression, they may predispose the cell to cancer and are thus termed oncogenes. Usually multiple oncogenes, along with mutated apoptotic or tumor suppressor genes will all act in concert to cause cancer. Since the 1970s, dozens of oncogenes have been identified in human cancers.

The word “proto-oncogene” refers to a normal gene that could become an oncogene due to mutation, increased expression, and/or other reasons. Proto-oncogenes code for proteins that help to regulate and differentiation. Proto-oncogenes are often involved in signal transduction and execution of mitogenic signals, usually through their protein products. Upon acquiring an activating mutation, a proto-oncogene can become an oncogene. Examples of proto-oncogenes include RAS, WNT, MYC, ERK, and TRK.

The phrase “MAP3K8 (COT; TPL2 in mouse) positive cancer” refers to a malignant tumor that expresses MAP3K8. The preferred biomarker, MAP3K8, includes polymorphic alleles existing in the human genome at the appropriate loci. Specifically, those alleles that have greater than 95% to the amino acid sequence of SEQ ID NO: 1 or the corresponding nucleotide sequence of SEQ ID NO:2 the exemplar gene for MAP3K8 (Accession No. D14497 Gene ID: 1326 (Seq. ID No: BAA03387.1 or NP_005195.2 or BAG36102.1 or CAG47079.1) Besson et al., 2017, Regulation of NF-KB by the P105-ABIN2-TPL2 Complex and RelAp43 during Rabies Virus Infection. PLoS Pathog. 13(10):e1006697; Wang et al., 2017 MiR-130b Attenuates Vascular Inflammation via Negatively Regulating Tumor Progression Locus 2 (Tpl2) Expression. Int Immunopharmacol.; 51:9-16; Jang, Kim, and Cha, 2017 Cot Kinase Plays a Critical Role in Helicobacter Pylori-Induced IL-8 Expression. J Microbiol. 55(4):311-317; Zhang et al., 2016 MiR-589-5p Inhibits MAP3K8 and Suppresses CD90+ Cancer Stem Cells in Hepatocellular Carcinoma. J Exp Clin Cancer Res. 35(1):176; Ballester et al., 1997 Cot Kinase Regulation of IL-2 Production in Jurkat T Cells. J Immunol. 159(4):1613-8; Salmeron et al., 1996 Activation of MEK-1 and SEK-1 by Tpl-2 Proto-Oncoprotein, a Novel MAP Kinase Kinase Kinase. EMBO J. 15(4):817-26. This gene is an oncogene that encodes a member of the serine/threonine protein kinase family. The encoded protein localizes to the cytoplasm and can activate both the MAP kinase and JNK kinase pathways. This gene may also utilize a downstream in-frame translation start codon, and thus produce an isoform containing a shorter N-terminus. The shorter isoform has been shown to display weaker transforming activity. Alternate splicing results in multiple transcript variants that encode the same protein. This gene encodes a protein containing 467 amino acids with a predicted molecular mass of 52,925 Da. It forms a ternary complex with NFKB1/p105 and TNIP2. It interacts with NFKB1; the interaction increases the stability of MAP3K8, but inhibits its MEK phosphorylation activity, whereas loss of interaction following LPS stimulation leads to its degradation. It interacts with CD40 and TRAF6; the interaction is required for ERK activation. It interacts with KSR2; the interaction inhibits ERK and NF-kappa-B activation.

The terms “identical” or percent “identity,” in the context of two or more polynucleotide or polypeptide sequences, refer to two or more sequences or subsequences that are the same or have a specified percentage of amino acid residues or nucleotides that are the same, when compared and aligned for maximum correspondence, as measured using a sequence comparison algorithms. A greater than 95% nucleotide or amino acid residue identity, is determined by comparing and aligning sequences for maximum correspondence, as measured by using the following sequence comparison algorithm. For purposes of this invention, sequence identity is determined using the BLAST programs with the default parameters. For example, parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

The phrase “COT inhibitor” refers to a compound that inhibits the kinase activity of COT, that is the ability of COT to catalyze the transfer of a gamma phosphate group from ATP to a serine or threonine residue in a protein or polypeptide that is a substrate of COT. Many inhibitors of protein kinases work by a similar principle of inhibiting the transfer of a gamma phosphate group from ATP to a serine or threonine, or in some cases a tyrosine, in the protein substrate of the kinase. COT inhibitor can refer to a compound that inhibits the binding of ATP to COT. COT inhibitor also refers to a compound that inhibits the protein-protein interaction between COT and any native interacting protein and thus inhibiting COT activity. This inhibition of the protein-protein interaction may be due to direct inhibition at the site of binding or by binding elsewhere in the protein that results in a conformational change that inhibits the interaction. COT inhibitor may interact with another protein that in turn binds to COT and/or binds to the substrate of COT, and thereby inhibits the kinase activity of COT. COT inhibitor may refer to any compound that might operate as an allosteric inhibitor of COT.

Anaplastic Lymphoma Kinase (ALK) is a receptor protein tyrosine kinase. The phrase “ALK fusion” refers to a fusion of a portion of the ALK polypeptide containing its tyrosine kinase activity with a variety of other polypeptides. For example, ALK commonly forms a fusion with a portion of the protein known Echinoderm Microtubule-Associated Protein-like 4, producing a fusion protein that is referred to as “EML4-ALK” or “ALK fusion.” ALK fusion is often found in patients with advanced NSCLC. ALK fusion can be inhibited by the drug Crizotinib, a small molecule inhibitor of the tyrosine kinases ALK, ROS1, and MET, as well as by additional drugs such as Alectinib, Ceritinib, Brigatinib and Lorlatinib. Unlike the case of the COT inhibitor discussed above, Crizotinib inhibits transfer of the gamma phosphate group from ATP to a tyrosine in the substrate protein of the ALK fusion.

The phrase “growth factor” (GF) refers to a naturally occurring substance capable of stimulating cellular growth, proliferation, healing, and cellular differentiation. Usually it is a protein or a lipid. Growth factors are important for regulating a variety of cellular processes. Growth factors typically act as signaling molecules between cells. Examples are cytokines and hormones that bind to specific receptors on the surface of their target cells.

The phrase “growth factor receptor” (GFR) refers to a protein whose function is to interact with a GF outside of the cell or on the cell surface, and initiate a signal processing system within the cell that may result in a response from the cell, such as cellular growth, proliferation, healing, or cellular differentiation. A GFR is often a transmembrane protein that is on the surface of the cell. GFRs often are transmembrane proteins that consist of an extracellular, ligand binding domain, one or more transmembrane domains, and an intracellular domain. The intracellular domain may contain an enzymatic activity, or may associate with other proteins that are enzymes, or both. For example, some GFRs are Receptor Tyrosine Kinases (RTK), that is the protein contains an extracellular domain(s) that bind a GF, one or more transmembrane domains, and one or more cytoplasmic domains. One or more of the cytoplasmic domains contains a tyrosine kinase enzymatic activity. RTKs can consist of one or more polypeptides.

The phrase “growth factor receptor inhibitor” (GFRI) (also known as growth factor blocker, growth factor receptor blocker, growth factor antagonist, growth factor receptor antagonist) are compounds that reduce the functioning of GFRs in cells. There are many mechanisms by which a GFRI can function. A few examples are provided here:

A GFRI can interfere with binding of the growth factor to the corresponding growth factor receptors; or can interfere with the receptor dimerization; or interfere with receptor auto-phosphorylation, interfere with phosphorylation of its partner dimer, or interfere with the GFR binding to downstream associating proteins. GFRI can target many types of GFR, including the EGFR, receptors for vascular endothelial growth factor, receptor for Hepatocyte Growth Factor, receptors fibroblast growth factors, cKIT and others.

The phrase “Mitogen activated protein kinase kinase (MPKK or MP2K) refers to a serine/threonine protein kinase, such as MEK1 or MEK2. MPKK are often considered part of signaling pathway that can lead from signaling starting at the plasma membrane by RAS and RAF, passing through a kinase cascade that may include MPKK, and leading to Extracellular signal-regulated kinases (ERK), such as ERK1 and/or ERK2. This signaling pathway is sometimes referred to as the “MAPK/ERK pathway.”

The phrase “MEK inhibitor” (MEKI) refers to compound that can inhibit the ability of MPKK to phosphorylate one or more of its substrate proteins. A MEKI can be used to affect the activity of MAPK/ERK pathway, which is often overactive in some cancers. Hence MEK inhibitors have potential for treatment of some cancers. Some examples of cancers that potentially will be treated with MEK inhibitors include melanomas containing mutations in BRAF, and colorectal cancers containing mutations in KRAS and BRAF.

KRAS is member of the family of small GTPases and can act as a switch in a signaling pathway controlling the proliferation, differentiation, and/or other behaviors of some cell types. The KRAS gene is often considered to be one of the most frequently or even the most frequently mutated gene in fatal cancers.

The phrase “KRAS inhibitor” refers to a compound that reduces the ability of KRAS to perform its signaling function. KRAS inhibitors can potentially be used to reduce the ability of abnormally active KRAS protein encoded by a mutated KRAS gene to cause the abnormal proliferation in a cancerous cell. There are many potential mechanisms

The RET proto-oncogene encodes a receptor tyrosine kinase for members of the of extracellular signaling molecules. RET gain of function mutations that are oncogenic are associated with the development of various types of human cancer, including medullary thyroid carcinoma, multiple endocrine neoplasia's type 2A and 2B, pheochromocytoma, parathyroid hyperplasia, and 1-2% of NSCLC patients.

The phrase “RET inhibitor” refers to a compound that reduces the ability of an oncogenic RET protein to cause activation of downstream signaling.

ROS1 is a proto-oncogene that encodes the ros1 receptor tyrosine kinase with structural similarity to the ALK protein described above. gene rearrangement events involving ROS1 have been described in NSCLC and other cancers.

The phrase “ROS1 fusion inhibitor” refers to a compound that reduces the ability of an oncogenic form of ROS1 fusion to induce cancerous behavior in a cancer cell. ROS1. Compounds that inhibit the tyrosine kinase enzymatic activity of ROS1 have been developed for ALK fusion and ROS1 fusions. Some of these compounds have been administered to patients with tumors that contain cells that express ALK fusion or ROS1 fusion and in some cases these compounds have been beneficial to these patients. FDA approved Inhibitors to ROS1 fusion and/or ALK fusion include Crizotinib, Alectinib, Ceritinib, Brigatinib and Lorlatinib.

Tyrosine-protein phosphatase non-receptor type 11 (PTPN11) also known as protein-tyrosine phosphatase 1D (PTP-1D), SHP-2, or protein-tyrosine phosphatase 2C (PTP-2C) is an enzyme that in humans is encoded by the PTPN11 gene. PTPN11 is a protein tyrosine phosphatase (PTP) and is also known as “SHP2.” Activating Shp2 mutations have also been detected in neuroblastoma, melanoma, acute myeloid leukemia, breast cancer, lung cancer, colorectal cancer. Recently, a relatively high prevalence of PTPN11 mutations (24%) were detected by next-generation sequencing in a cohort of NPM1-mutated acute myeloid leukemia patients, although the prognostic significance of such associations has not been clarified. These data suggests that Shp2 may be a proto-oncogene. However, it has been reported that PTPN11/Shp2 can act as either tumor promoter or suppressor.

The phrase “SHP-2 inhibitor” refers to a compound that can reduce the ability of an oncogenic form of SHP-2 to promote the cancerous behavior of cancer cells.

Cell cycle proteins, also known as Cyclin-Dependent Kinases (CDKs), are a family of serine-threonine protein kinases that regulate the progression through the cell cycle. CDKs bind the regulatory protein cyclin and, together as a heterodimer, phosphorylate their appropriate substrates for progressing through a particular cell cycle phase. In many cancers, CDKs are overactive or CDK-inhibiting proteins are not functional. Therefore these proteins may have oncogenic activity. CDK inhibitors are used to treat cancers by preventing over proliferation of cancer cells.

The phrase “cell cycle inhibitor” refers to a compound that reduces the function of a broad types of CDKs or a specific type of CDK. The FDA has approved several inhibitors of CDK4 and CDK 6, which include Ribociclib, Palbociclib and Abemaciclib.

DNA repair proteins are cell proteins that participate in restoring damaged DNA to prevent cell death. Mutations in DNA repair proteins, such as BRCA1/2 or PALB2, cause an increase in mutated proteins and can eventually cause breast cancer, ovarian cancer or other cancers. The family of DNA repair proteins, in part, consist of proteins called Poly ADP Ribose Polymerase (PARP) that participate in repairing double-strand breaks in DNA.

The phrase “PARP inhibitors” refers to compounds that cause multiple double-strand breaks to form in this way. In tumors with BRCA1/2 or PALB2 mutations, these double strand breaks cannot be efficiently repaired, leading to the death of the cells. Normal cells that don't replicate their DNA as often as cancer cells, and that lack any mutated BRCA1 or BRCA2, still have homologous repair operating, which allows them to survive the inhibition of PARP. In contrast, cancer cells harboring mutated BRCA1/2 or PALB2, PARP inhibition interferes with replication, causing cell death while not affecting non-cancerous cells. The FDA has approved several PARP inhibitors which include Olaparib, Rucaparib, Niraparib, Talazoparib. Several other inhibitors are under development, including INO-1001, Pamiparib, E7449 and Iniparib.

The phrase “targeted therapy” refers to one (or more, singly or in combination) compound, molecule, element, drug, medicine, regimin, food, pharmaceutical, biologic, protein, antibody, protein fusion, virus, recombinant virus, immunological agent, cell, organism, or any other means of treatment that has as its goal the reduction and/or elimination of cancer in a patient, and that has a mechanism of action that is believed to work by acting on one or more of the molecular and/or cellular mechanisms underlying the particular cancer from which the patient is suffering. All forms of immune therapy and immunotherapy are included in this definition.

The phrase “therapeutic resistance” refers to the following phenomenon: When a patient with a cancer is initially treated with a targeted therapy drug, the cancer at first responds. For example, if the patient contains a malignant tumor that is visible by an imaging technique, the size of the tumor may shrink in response to the targeted therapy. However, after this initial response, the malignant tumor may eventually regrow and/or a new tumor of the same type may grow in a new location in the patient's body. This regrown or new tumor will continue to grow despite continued administration of the original targeted therapy drug. The cells in this malignant tumor are said to have acquired “therapeutic resistance”.

The phrase “therapeutic resistance” may also refers to innate therapeutic resistance: When a tumor expresses a protein that its sole expression results with reduced efficacy of the chemotherapeutic drug. This could range from a protein activating a signaling pathway downstream from the target of treatment, activating a signaling pathway parallel to the one targeted by treatment or even a liver metabolizing enzyme that will directly or indirectly reduce the half-life of the drug.

There are several broad categories by which cancer cells gain resistance to a targeted therapy drug. In one broad category, referred to as “On-Target” an additional mutation occurs in the gene whose product was originally targeted by the targeted therapy. This can be a compound mutation causing a conformational change that disrupts the structure of the binding site or an additional oncogenic mutation causing the target to regain oncogenic activity. Cells harboring the compound mutation continue to proliferate while the other cells die due to drug treatment. In a second broad category, referred to as “Off-Target,” an alteration occurs in a different gene. This alteration may be an overexpression or mutation. The mutation is most often a gain of function mutation that activates an oncogenic signaling pathway that bypasses the pathway inhibited by the original targeted therapy. Many other mechanisms, combinations and permutations are also possible. As with on-target mutations, the cells harboring the off-target alteration will survive and proliferate, while the original cells are inhibited and usually die. In both cases, the surviving cells generate an aggressive tumor that can lead to relapse and death of the patient. Overcoming Therapeutic Resistance requires a new treatment approach.

1. Cancers that Originate from Oncogenes

Oncogenes are genes that have the potential to cause cancer. These genes when mutated stimulate cell proliferation (overgrowth) causing growth of cancer cells. Mutations in genes that become oncogenes can be inherited, or caused by being exposed to substances in the environment, or due to malfunction of the cell's DNA repair system may cause cancer. Activated oncogenes can cause cells designated for apoptosis (cell death) to survive and proliferate instead. If normal genes promoting cellular growth, are up-regulated through mutation (gain of function mutation), they will predispose the cell to cancer and are thus termed oncogenes. Usually multiple oncogenes, along with mutated apoptotic or tumor suppressor genes will all act in concert to cause cancer. Dozens of oncogenes have been identified in human cancers.

A. Growth Factor Receptors:

A growth factor is a naturally occurring substance capable of stimulating its receptor by which it regulates cellular growth, proliferation, healing, cellular differentiation and other processes. Some growth factor receptors are localized to the plasma membrane, where they are activated by the growth factor at the extracellular surface, which causes the initiation of the growth factor receptor's signaling, which is transmitted to the cell's cytosol and nucleus. Oncogenic growth factor receptors are those receptors for which their over expression or specific mutation will contribute to the cell becoming a cancer cell. Good examples for these receptors are the family of epidermal growth factor receptors (EGFR), fibroblast growth factor receptors (FGFR), Hepatocyte growth factor receptor (cMET), Platelet-derived growth factor Receptors (PDGFR), Transforming growth factors receptors (TGF-α and TGF-β), Tumor necrosis factor receptors (TNFR1 and TNFR2), and others.

B. KRAS:

KRAS is a protein that acts as an on/off switch in cell signaling. When it functions normally, it transmits and propagates the signal of growth factors through their receptors to control cell proliferation, differentiation and other processes. It transmits its signal through two main signaling pathways, the MAPK and PI3 Kinase pathways. When it is mutated in specific sites (mainly amino acid residues 12, 16 and 61), negative signaling by KRAS is disrupted and the protein is constitutively on. Thus, cells can continuously proliferate and often develop into cancer.

The oncogenic KRAS protein is frequently present in various malignant tumors, including lung adenocarcinoma, mucinous adenoma, ductal carcinoma of the pancreas and colorectal cancer. Much effort has been invested to develop a drug that specifically inhibits oncogenic KRAS. To date, no drug has been approved by the FDA to inhibit KRAS. Cancer patients with an oncogenic KRAS generally suffer from a poor prognosis.

C. The ALK Fusion Gene:

A group of patients with non-small cell lung cancer (NSCLC) have tumors that contain an inversion in chromosome 2 that brings together the 5′ end of the echinoderm microtubule-associated protein-like 4 (EML4) gene with the 3′ end of the anaplastic lymphoma kinase (ALK) gene, resulting in the novel fusion oncogene EML4-ALK. This genetic rearrangement creates a fusion oncogenic protein in NSCLC. It is found in approximately 7 percent of NSCLC patients.

Patients with EML4-ALK fusion do not possess mutations in EGFR and KRAS that are common in lung cancer. This group is usually of younger age and are non-smokers.

Several targeted therapy drugs for this disease are FDA approved and include Crizotinib, Brigatinib, Alectinib, Ceritinib, and Lorlatinib. Treatment is initiated with Crizotinib and is generally continued until there is evidence of disease progression. While Crizotinib is highly active in patients with ALK-positive NSCLC, almost all patients develop resistance to the drug, typically within the first few years of treatment. Upon progression, which is due to the development of resistance to the drug, treatment with a next-generation EML4-ALK fusion inhibitor or with standard chemotherapy may be administered.

D. RET Fusion Genes

RET is a proto-oncogene localized to chromosome 10, which encodes the protein RET, a receptor tyrosine kinase (RTK). Through alternative splicing RET encodes three protein isoforms: RET9, RET32, and RET51. RET receives signals from the glial cell-derived neurotrophic factor (GDNF) family of ligands. Unlike many other RTKs, RET signaling requires co-receptors that are bound to RET. RET signaling occurs through the MAPK, PI3K and STAT3 pathways, which play key roles in kidney and nervous system development, neuronal survival and differentiation, and maintenance of spermatogonial stem cells.

RET mutations play an important role in thyroid carcinoma, papillary thyroid carcinoma, and others. RET chromosomal rearrangements generate an oncogene that is a result of inversion in chromosome 10 fusion that participates in many cancers, especially in lung cancer (NSCLC). RET aberrations were identified to account for 1.8% of diverse cancers, with RET fusions accounting for 0.6% of cases. RET fusions were mutually exclusive with MAPK signaling pathway alterations. Treatments of thyroid cancer patients harboring RET fusion include multi-kinase inhibitors (Cabozantinib, lenvatinib and vandetanib). Similar drugs are currently in trials for lung cancer, with additional drugs under clinical trials (LOXO-292 and RXDX-105).

E. ROS1 Fusion Gene

ROS1, a proto-oncogene, is highly expressed in a variety of tumor cells, belongs to the Sevenless subfamily of tyrosine kinase receptor genes. The protein may function as a growth or differentiation factor receptor.

ROS1 protein has a structural similarity to the anaplastic lymphoma kinase (ALK) protein. The exact role of the ROS1 protein i in development, as well as its normal physiologic ligand, has not been defined. Nonetheless, gene rearrangement events involving ROS1 in lung and other cancers are responsive to crizotinib. Such gene rearrangements result in cancers including glioblastoma multiforme (GBM), non-small cell lung cancer (NSCLC), cholangiocarcinoma, ovarian cancer, gastric cancer and colorectal cancer.

Crizotinib is the standard of care for metastatic ROS1 positive non-small cell lung cancer (NSCLC). In clinical trials, crizotinib was shown to be initially effective in 70-80% of patients. Additional FDA approved drugs serve as second line to overcome resistance to crizotinib or to overcome brain metastasis. These include ceritinib that overcome brain metastasis, cabozantinib, brigatinib, lorlatinib, Entrectinib (in trials), and entrectinib that showed 78% objective response rate (ORR) and duration of response of 28.6 months. Some ALK inhibitors do not possess clinical benefit in ROS1 positive patients. These include foretinib and alectinib.

### 2. Detecting Oncogene Originating Cancers

This invention depends on the physician having a clear picture of the underlying genetic basis the cancer afflicting the patient. Below is a listing of the available assays for determining the genetic bases for cancers originating from oncogenes. This list, as well as all other lists in this patent, is meant to serve as only as a list of examples. It does not include all such assays that may be available currently. It certainly cannot include other assays that may be developed in the future.

A. Detection of an Overexpression of Oncogene in Tumor Samples by Immunohistochemistry

Detection typically begins with a biopsy harvested from the malignant tissue. The tissue is formalin-fixed and paraffin-embedded (FFPE). The sample is sectioned with a microtome to yield a section that is of an appropriate thickness for microscopy. The section is mounted on a glass slide. Typically, it is first stained with one or more conventional histological stains, such as hematoxylin and eosin. The embedded paraffin section is then subjected to a blocking step. Next an appropriate primary antibody (for instance, rabbit or mouse, that identifies the target gene) is applied. After a wash step a secondary antibody pre-linked to horse radish peroxidase (HRP) is applied. Subsequently, an HRP substrate is applied and excess is washed, the sample dehydrated, and covered with mounting media and a coverslip. The sample is then ready for microscopic analyses.

The following FDA terminology is used herein:

NDA stands for New Drug Application

BLA stands for Biological License Application

PMA stands for Premarket Approval

510 (k) refers to a type of Premarket Approval for Class II medical devices

HDE stands for Humanitarian Device Exemption.

Examples of FDA-approved companion diagnostic immunohistochemistry (IHC) Assays include the following:

| | | | Trade Name |
|---|---|---|---|
| | PMA/510(k)/ | Diagnostic | (Generic)- |
| Diagnostic Name | HDE | Manufacturer | NDA/BLA |
| PD-L1 (SP142) | P160002/S006 | Ventana | Non-small cell lung |
| Polyclonal anti PD-L1 for IHC | | Medical | cancer and urothelial |
| | | Systems, Inc. | carcinoma |
| | | | Tecentriq |
| | | | (atezolizumab)- |
| | | | NDA 761034/S012 |
| PATHWAY anti-Her2/neu | P990081/S001- | Ventana | Breast cancer |
| (4B5) Rabbit Monoclonal | S028 | Medical | Herceptin |
| Primary Antibody | | Systems, Inc. | (trastuzumab)- |
| IMMUNOHISTOCHEMISTRY, | | | BLA 103792 |
| antibody | | | |
| VENTANA ALK (D5F3) CDx | P140025/S006 | Ventana | Non-small cell lung |
| Assay | | Medical | cancer |
| IHC for ALK expressing cells | | Systems, Inc. | Zykadia |
| | | | (ceritinib)- |
| | | | NDA 205755 |
| | | | Xalkori |
| | | | (crizotinib)- |
| | | | NDA 202570 |
| | | | Alecensa (alectinib)- |
| | | | NDA 208434 |
| Dako EGFR pharmDx Kit | P030044/S002 | Dako North | Colorectal cancer |
| IHC for EGFR | | America, Inc. | Erbitux |
| | | | (cetuximab)- |
| | | | BLA 125084 |
| | | | Vectibix |
| | | | (panitumumab)- |
| | | | BLA 125147 |
| Dako c-KIT pharmDx | P040011 | Dako North | Gastrointestinal |
| cKIT IHC overexpression | | America, Inc. | stromal tumors |
| | | | Gleevec (imatinib |
| | | | mesylate) |
| | | | NDA 021335 |
| | | | Glivec (imatinib |
| | | | mesylate) |
| | | | NDA 021588 |
| PD-L1 IHC 22C3 pharmDx | P150013/S011 | Dako North | Non-small cell lung |
| IHC for PD-L1 | | America, Inc. | cancer, gastric or |
| | | | gastroesophageal |
| | | | junction |
| | | | adenocarcinoma, |
| | | | cervical cancer, and |
| | | | urothelial carcinoma |
| | | | Keytruda |
| | | | (pembrolizimab)- |
| | | | BLA 125514 |
| HercepTest | P980018/S018 | Dako | Breast cancer |
| IHC her2 overexpression | | | Denmark A/S Herceptin |
| | | | (trastuzumab)- |
| | | | BLA 103792 |
| | | | Perjeta |
| | | | (pertuzumab)- |
| | | | BLA 125409 |
| | | | Kadcyla (ado- |
| | | | trastuzumab |
| | | | emtansine)- |
| | | | BLA 125427 |
| | | | Gastic and |
| | | | gastroesophogeal |
| | | | cancer |
| | | | Herceptin |
| | | | (trastuzumab)- |
| | | | BLA 103792 |
| Bond Oracle HER2 IHC | P090015 | Leica | Breast cancer |
| System | | Biosystems | Herceptin |
| IHC | | | (trastuzumab)- |
| | | | BLA 103792 |

B. Detection of Overexpression of an Oncogene in Tumor Samples by Chromogenic In Situ Hybridization (CISH)

CISH, a highly sensitive method to identify the overexpression of an oncogene through identification of gene amplification using horse radish peroxidase (HRP) as a reporter. The major advantage of CISH is in its superior sensitivity compared to FISH. Slides made from formalin-fixed, paraffin-embedded (FFPE) patient tumor samples are deparaffinized and rehydrated by heating to 80° C. Subsequently, the sample goes through a protease enzymatic digestion protocol to remove non-nucleic acids from the sample and expose the sample DNA. The DNA probe coding for a fragment of the gene to be quantified is then hybridized to the sample. The DNA probe may be pre-linked to either Biotin, Digoxigenin or HRP. Following hybridization, a wash step is performed to remove unbound probe, followed by a blocking step to prevent nonspecific protein binding sites. Finally, a staining step is performed. If horseradish peroxidase is used, the sample must be incubated in hydrogen peroxide to suppress endogenous peroxidase activity. If digoxigenin was used as a probe label, an anti-digoxigenin fluorescein primary antibody followed by an HRP-conjugated anti-fluorescein secondary antibody are then applied. If biotin was used as a probe label, non-specific binding sites must first be blocked using bovine serum albumin (BSA). HRP-conjugated streptavidin is used for detection. HRP then converts its substrate diaminobenzidine (DAB) into an insoluble brown product, which can be detected using bright-field microscope.

Examples of FDA-approved companion diagnostic CISH protocols include the following:

| | PMA/510(k)/ | Diagnostic | Trade Name (Generic)- |
|---|---|---|---|
| Diagnostic Name | HDE | Manufacturer | NDA/BLA |
| SPOT-LIGHT HER2 | P050040/S001- | Life | Breast cancer |
| CISH Kit | S003 | Technologies | Herceptin (trastuzumab)- |
| CISH gene | | Corporation | BLA 103792 |
| amplification | | | |
| HER2 CISH | P100024 | Dako Denmark | Breast cancer |
| pharmDx Kit | | A/S | Herceptin (trastuzumab)- |
| Her2 gene | | | BLA 103792 |
| amplification | | | |
| INFORM HER2 Dual | P100027 | Ventana | Breast cancer |
| ISH DNA Probe | | Medical | Herceptin (trastuzumab)- |
| Cocktail | | Systems, Inc. | BLA 103792 |
| Gene amplification | | | |

C. Detection of an Overexpression of an Oncogene in a Tumor Samples by Fluorescence In Situ Hybridization (FISH)

In oncology, fluorescence in situ hybridization (FISH) is a test that “maps” or localizes the genetic material in the tumor cells, including specific genes or portions of genes. Therefore, the protocols can detect a deletion, an increased copy number of a given gene, translocation of a gene or part of it to be fused to another gene or translocated to another chromosome. In specific genes these type of aberration may cause them to be oncogenes. Such genes include, but are not limited to, HER2, EGFR, ALK, ROS, BRAC1/2 and others.

In general, formalin-fixed, paraffin-embedded (FFPE) patient tumor slides are deparaffinized and rehydrated, and subsequently are protease treated to remove proteins and peptides from the sample and expose the sample DNA. Therefore, a DNA probe coding for a fragment of the gene to be quantified is then hybridized to the sample. The probe is tagged directly with fluorophores, or with targets for antibodies or with biotin. The probe is then applied to the slide so it can bind to the chromosomal DNA and incubated to enable hybridizing. Following several wash steps to remove all unhybridized or partially hybridized probes, the sample can be visualized and quantified using a fluorescent microscope. Analyses will enable the detection of gene deletion, increased copy number or several types of translocations.

Examples of FDA-approved companion diagnostic FISH protocols include the following:

| | PMA/510(k)/ | Diagnostic | Trade Name (Generic)- |
|---|---|---|---|
| Diagnostic Name | HDE | Manufacturer | NDA/BLA |
| Vysis CLL FISH | P150041 | Abbott | B-cell chronic lymphocytic |
| Probe Kit | | Molecular, | leukemia |
| Fluorescence in situ | | Inc. | Venclexta (venetoclax) |
| hybridization. Genomic | | | NDA 208573 |
| deletion of P53 | | | |
| Vysis ALK Break | P110012 | Abbott | Non-small cell lung cancer |
| Apart FISH Probe Kit | | Molecular Inc. | Xalkori (crizotinib) |
| ALK fusion detection | | | NDA 202570 |
| through FISH | | | |
| PDGFRB FISH for | H140005 | ARUP | Myelodysplastic |
| Gleevec Eligibility in | | Laboratories, | syndrome/myeloproliferative |
| Myelodysplastic | | Inc. | disease |
| Syndrome/ | | | Gleevec (imatinib |
| Myeloproliferative | | | mesylate)-NDA 021335 |
| Disease (MDS/MPD) | | | |
| FISH gene | | | |
| rearrangement | | | |
| INFORM HER-2/neu | P940004 | Ventana | Breast cancer |
| Genomic gene | | Medical | Herceptin (trastuzumab)- |
| amplification FISH | | Systems, Inc. | BLA 103792 |
| PathVysion HER-2 | P980024 | Abbott | Breast cancer |
| DNA Probe Kit | | Molecular Inc. | Herceptin (trastuzumab)- |
| Genomic gene | | | BLA 103792 |
| amplification FISH | | | |
| HER2 FISH pharmDx | P040005/S009 | Dako | Breast cancer |
| Kit | | Denmark A/S | Herceptin (trastuzumab)- |
| FISH for HER2 gene | | | BLA 103792 |
| amplification | | | Perjeta (pertuzumab)- |
| | | | BLA 125409 |
| | | | Kadcyla (ado- |
| | | | trastuzumab emtansine)- |
| | | | BLA 125427 |
| | | | Gastic and gastroesophogeal |
| | | | cancer |
| | | | Herceptin (trastuzumab)- |
| | | | BLA 103792 |
| Vysis ALK Break | P110012 | Abbott | Non-small cell lung cancer |
| Apart FISH Probe Kit | | Molecular Inc. | Xalkori (crizotinib)- |
| ALK fusion detection | | | NDA 202570 |
| through FISH | | | |

D. Detection of Mutations and Overexpression of an Oncogene in a Tumor Samples by DNA Sequencing, Using Sanger and Next Generation Sequencing

DNA aberrations which include point mutations, insertions, deletions and/or other alterations, may cause change of amino acids, frame-shifts, and fusions, that may transform a proto-oncogene into an oncogene. To identify the mutated oncogene and, in turn, the correct choice of drug treatment, the mutations need to be identified. This identification can be performed by DNA sequencing. In this process the sequence of nucleotides in DNA is determined. Computational methods are then used to process this data and determine mutations and other alterations in the DNA sequence. The process of identification can be done by the Sanger sequencing method for a single gene at a time or by next generation sequencing (NGS) in which parallel sequencing of a large number of genes is performed.

A brief description of the Sanger sequencing method is as follows: (i) the gene of interest is amplified by the PCR reaction and is cleaned up from residual primers oligos and other contaminants; (ii) cycle amplification, which includes fluorescently labeled modified nucleotides whose incorporation terminates the polymerase reaction, is carried out using four different fluorophores (of different colors) that are used to distinguish between the nucleotides which thereby indicate their nucleotide position; and (iii) the mix of chains generated, each end labeled by a different nucleotide and color, are loaded onto a DNA sequencer that separates the chains by their size and determines their fluorophore color, enabling the determination of the DNA sequence.

Modern sequencing is done by Next Generation Sequencing. In principle, the concept behind NGS technology is similar to capillary electrophoresis sequencing. Initially, a DNA polymerase catalyzes the incorporation of fluorescently labeled deoxyribonucleotide triphosphates (dNTPs) into a DNA template strand during sequential cycles of DNA synthesis. During each cycle, at the point of incorporation, the nucleotides are identified by fluorophore excitation. The critical difference is that, instead of sequencing a single DNA fragment, NGS extends this process across millions of fragments in a massively parallel fashion. A general description follows:

(i) Library Preparation: random fragmentation of the DNA or cDNA sample, followed By adapter ligation followed by PCR amplification and purification.

(ii) Cluster Generation: the library is loaded into a flow cell where fragments are captured on a lawn of surface-bound oligos complementary to the library adapters. Each fragment is then amplified into distinct, clonal clusters through bridge amplification. When cluster generation is complete, the templates are ready for sequencing.

(iii) Sequencing: using the Illumina SBS technology (FDA approved) which uses reversible terminator based method that detects single bases as they are incorporated into DNA template strands.

Data Analysis: the newly identified sequence reads are aligned to a reference genome. Following alignment, many types of data analysis are possible, such as single nucleotide polymorphism or insertion-deletion identification, read counting for RNA methods, phylogenetic or metagenomic analysis, and more.

Examples of FDA-approved companion diagnostic approved sequencing-based protocols include the following:

| | PMA/510(k)/ | Diagnostic | Trade Name (Generic)- |
|---|---|---|---|
| Diagnostic Name | HDE | Manufacturer | NDA/BLA |
| BRACAnalysis | P140020/S016 | Myriad | Breast Cancer |
| CDxMultiplex PCR | | Genetic | Lynparza (olaparib)- |
| followed by sanger | | Laboratories, | NDA 208558 |
| seq to detect SNV/ | | Inc. | Talzenna (talazoparib) |
| Indel's | | | NDA 211651 |
| | | | Ovarian Cancer |
| | | | Lynparza (olaparib)- |
| | | | NDA 208558 |
| | | | Rubraca (rucaparib) |
| | | | NDA 209115 |
| FoundationOne CDx | P170019 | Foundation | Non-small cell lung cancer |
| NGS, 326 genes, | | Medicine, Inc. | Gilotrif (afatinib)- |
| SNV, copy number, | | | NDA 201292 |
| gene rearrangements, | | | Iressa (gefitinib)- |
| microsatellite | | | NDA 206995 |
| instability (MSI) and | | | |
| tumor mutational | | | Tarceva (erlotinib)- |
| burden (TMB) | | | NDA 021743 |
| Tagrisso (osimertinib) | | | |
| | | | NDA 208065 |
| | | | Alecensa (alectinib)- |
| | | | NDA 208434 |
| | | | Xalkori (crizotinib)- |
| | | | NDA 202570 |
| | | | Zykadia (ceritinib)- |
| | | | NDA 205755 |
| | | | Tafinlar (dabrafenib)- |
| | | | NDA 202806 in |
| | | | combination with Mekinist |
| | | | (trametinib)-NDA 204114 |
| | | | Melanoma |
| | | | Tafinlar (dabrafenib)- |
| | | | NDA 202806 |
| | | | Zelboraf (vemurafenib)- |
| | | | NDA 202429 |
| | | | Mekinist (trametinib)- |
| | | | NDA 204114 or Cotellic |
| | | | (cobimetinib)- |
| | | | NDA 206192 in |
| | | | combination with Zelboraf |
| | | | (vemurafenib)- |
| | | | NDA 202429 |
| | | | Breast cancer |
| | | | Herceptin (trastuzumab)- |
| | | | BLA 103792 |
| | | | Perjeta (pertuzumab)- |
| | | | BLA 125409 |
| | | | Kadcyla (ado-trastuzumab |
| | | | emtansine)-BLA 125427 |
| | | | Colorectal cancer |
| | | | Erbitux (cetuximab)- |
| | | | BLA 125084 |
| | | | Vectibix (panitumumab)- |
| | | | BLA 125147 |
| | | | Ovarian cancer |
| | | | Rubraca (rucaparib)- |
| | | | NDA 209115 |
| Oncomine Dx Target | P160045 | Life | Non-small cell lung cancer |
| Test | | Technologies | Tafinlar (dabrafenib)- |
| NGS. Mutations and | | Corporation | NDA 202806 |
| fusions | | | Mekinist (trametinib)- |
| | | | NDA 204114 |
| | | | Xalkori (crizotinib)- |
| | | | NDA 202570 |
| | | | Iressa (gefitinib)- |
| | | | NDA 206995 |
| FoundationFocus | P160018 | Foundation | Ovarian cancer |
| CDxBRCA Assay | | Medicine, Inc. | Rubraca (rucaparib)- |
| BRCA mutation NGS | | | NDA 209115 |

E. Detection of Mutations and Overexpression of an Oncogene in a Tumor Samples by Either Reverse Transcription Polymerase Chain Reaction (RTPCR) or Real-Time Polymerase Chain Reaction (qPCR)

These two reaction are a variant of the polymerase chain reaction (PCR) used to detect gene expression. RT-PCR is used to qualitatively detect gene expression through the creation of complementary DNA (cDNA) transcripts from RNA, whereas qPCR (quantitative PCR) is used to quantitatively measure the amplification of DNA using fluorescent dyes. A general description of each of these methods is set forth below.

RT-PCR: One-step RT-PCR take mRNA targets (up to 6 kb) and subjects them to reverse transcription and then PCR amplification in a single test tube. In this assay, a mix with reverse transcriptase and the PCR system, such as Taq DNA Polymerase, and a proofreading polymerase is used. A mix of these two enzymes are mixed with dNTPs, primers, template RNA, and placed in the PCR thermal cycler machine. In the first cycle, the reverse transcriptase synthesizes the cDNA and is inactivated. In the next 40 to 50 cycles, the amplification of the target DNA segment takes place. This segment is specific to the gene to be measured such as HER2 or BRAF. Finally, the RT-PCR products are analyzed by gel electrophoresis.

qPCR: One-step qPCR combines reverse transcription and PCR in a single tube and buffer, using a reverse transcriptase along with a DNA polymerase. One-step RT-qPCR only utilizes sequence-specific primers. In this protocol, a soluble fluorescent dye is incorporated into the growing DNA chain. This enables monitoring at each cycle of PCR the amount of DNA amplified indicating on the initial amount of the gene to be measured. When the DNA is in the log linear phase of amplification, the amount of fluorescence increases above the background. This protocol is done using the same protocol as RT-PCR with the addition of a soluble fluorescent dye (e.g., Cyber green) that when incorporated into the growing DNA chain is fluorescent. This protocol requires a qPCR instrument that will measure the fluorescence in each tube every cycle. The rate of increase of fluorescence is used to quantitate the amount of starting DNA that is reverse transcribed from the mRNA.

Examples of FDA companion diagnostic approved RT-PCR and qPCR-based protocols include the following:

| Diagnostic | PMA/510(k)/ | Diagnostic | Trade Name (Generic)- |
|---|---|---|---|
| Name | HDE | Manufacturer | NDA/BLA |
| SPOT-LIGHT | P050040/S001- | Life | Breast cancer |
| HER2 CISH Kit | S003 | Technologies | Herceptin (trastuzumab)- |
| CISH gene | | Corporation | BLA 103792 |
| amplification | | | |
| HER2 CISH | P100024 | Dako | Breast cancer |
| pharmDx Kit | | Denmark A/S | Herceptin (trastuzumab)- |
| Her2 gene | | | BLA 103792 |
| amplification | | | |
| INFORM HER2 | P100027 | Ventana | Breast cancer |
| Dual ISH DNA | | Medical | Herceptin (trastuzumab)- |
| Probe Cocktail | | Systems, Inc. | BLA 103792 |
| Gene | | | |
| amplification | | | |
| cobas 4800 | P110020/S016 | Roche | Melanoma |
| BRAF V600 | | Molecular | Zelboraf (vemurafenib)- |
| Mutation Test | | Systems, Inc. | NDA 202429 |
| rtPCR for BRAF | | | Cotellic (cobimetinib)- |
| V600 Mutations | | | NDA 206192 in combination |
| | | | with Zelboraf (vemurafenib)- |
| | | | NDA 202429 |

### 3. Determining MAP3K8 (COT) Status

Once the genetic basis for the cancer has been determined, it is important to determine or confirm that the existing or future resistance to inhibitors directed to specific oncogenes can be evaluated. In this invention, that determination arises from the detection of an elevated level of MAP3K8, which is also named Cot (Cancer Osaka Thyroid) or Tpl-2 (Tumor progression locus-2). MAP3K8 is an oncogene that encodes a member of the serine/threonine protein kinase family. The encoded protein localizes to the cytoplasm and is required for lipopolysaccharide (LPS)-induced, TLR4-mediated activation of the MAPK/ERK pathway in macrophages, thus being critical for production of the proinflammatory cytokine TNF-alpha (TNF-alpha) during immune responses. MAP3K8 is involved in the regulation of T-helper cell differentiation and IFN-gamma expression in T-cells and in mediating host resistance to bacterial infection through negative regulation of type I interferon (IFN) production. In vitro, MAP3K8 activates the MAPK/ERK pathway in response to IL1 in an IRAK1-independent manner, leading to the up-regulation of IL8 and CCL4. MAP3K8 transduces the CD40 and TNFRSF1A signals that activate ERK in B-cells and macrophages, and thus is thought to play a role in the regulation of immunoglobulin production. MAP3K8 may also play a role in the transduction of TNF signals that activate JNK and NF-kappa-B in some cell types. In adipocytes, MAP3K8 activates the MAPK/ERK pathway in an IKBKB-dependent manner in response to IL1B and TNF, but not insulin, leading to induction of lipolysis. MAP3K8 plays a role in the cell cycle. Isoform 1 of MAP3K8 shows some transforming activity, although it is much weaker than that of the activated oncogenic variant.

MAP3K8 has been shown to be involved in inflammatory diseases, such as rheumatoid arthritis (RA), multiple sclerosis (MS), inflammatory bowel disease (IBD), diabetes, sepsis, psoriasis, mis-regulated TNFα expression, graft rejection and cancer.

Agents and methods that modulate the expression or activity of COT, or agents that synergize with COT inhibitors will be useful for preventing or treating such diseases.

The oncogenic activity of COT expressed in the target tissue will be assessed by a single or a combination of methodologies. Each method provides a score that is added together using a predefined and approved method. These methods include, but are not limited to Immunohistochemistry, sequencing (NGS, Sanger or other), qPCR and RTPCR, FISH, CISH or other methodologies that will enable quantitation of the following parameters: (i) level of expression, (ii) gene copy number or duplication, (iii) mutations or fusions, (iv) phosphorylation status of COT or phosphorylation of downstream signaling protein, and (vi) subcellular location which indicates on active form/conformation. For that objective, the diagnostic method may use tumor tissue biopsy, percutaneous biopsy, liquid biopsy of any form such as blood, serum, lymph (such as cerebrospinal fluid, urine and ascites fluid), saliva, urine, and serous fluids (pleural effusion and pericardial effusion).

### 4. Inhibitors of Oncogene Activity

This invention is directed to the use of a combination of COT inhibitors with known inhibitors of oncogenes, or proteins expressed from oncogenes, or inhibitors of cell cycle or DNA repair proteins, or any other inhibitor that is used as a cancer therapeutic drug, or any other compound that is used to treat cancer. Representative examples of these inhibitors are provided below according to class.

### A. Growth Factor Receptor Inhibitors

EGFR: Cetuximab, necitumumab, Erlotinib, Afatinib Lapatinib Gefitinib, panitumumab, vandetanib, osimertinib, neratinib

Her2: Neratinib, Trastuzumab, lapatinib, pertuzumab

cMET: Crizotinib, Cabozantinib, Glesatinib, Capmatinib, MK-2461

c-KIT: Imatinib, Dovitinib, Tivozanib, Amuvatinib, Telatinib

Vascular endothelial growth factor receptor (VEGFR): Sunitinib, Pazopanib, Axitinib, Regorafenib, Sorafenib, Lenvatinib

Platelet-derived growth factor receptor (PDGFR): Sunitinib, Pazopanib, Regorafenib, Sorafenib, Lenvatinib

B. Other Oncogene Inhibitors

KRAS inhibitors: (no current FDA approved drug to KRAS). The following are direct inhibitors: ARS-853; ARS-1620; AMG-510; and MTRX849 (Mutant-specific RAS inhibitors). The following are indirect inhibitors: Indirect inhibition: RO5126766 (Raf/MEK dual inhibitor); tipifarnib, lonafarnib, bms-214662 (Farnesyl transferase inhibitors); Deltarasin (KRAS-PDEδ inhibitors); Ras-Raf interaction inhibitors (sulindac-derived compounds); Kobe0065 and Kobe2602 (SOS binding inhibitors); SHP099, NSC87877 (SHP inhibitors)

MEK inhibitors: Cobimetinib, and Trametinib.

JAK inhibitors: Fedratinib, Tofacitinib, PF-06651600, Baricitinib, Filgotinib

ALK fusion inhibitors: crizotinib (Xalkori), alectinib (Alecensa), ceritinib (Zykadia), and brigatinib (Alunbrig), Lorlatinib, and Gilteritinib.

RET fusion genes or RET alone: Cabozantinib, LOXO-292, Alectinib, Lenvatinib, and Ponatinib

ROS1: fusion gene: Brigatinib, Cabozantinib, Ceritinib, Crizotinib

### 5. Inhibitors of COT

Provided below are several examples of known COT inhibitors and functional assays used to determine COT inhibitory activity. COT inhibitors of this invention include a structurally diverse universe of compounds, and based on the teachings of the present invention, these compounds can be tested for their ability to inhibit COT using a number of assays.

The compounds described below were originally developed to treat inflammatory diseases such as arthritis wherein inflammation contributes to deterioration of synovial cartilage. Inhibition of TNF alpha secretion supports inhibition of inflammation.

A. Quinoline-3-carbonitriles as COT Inhibitors (1,7-naphthyridine-3-carbonitrile)

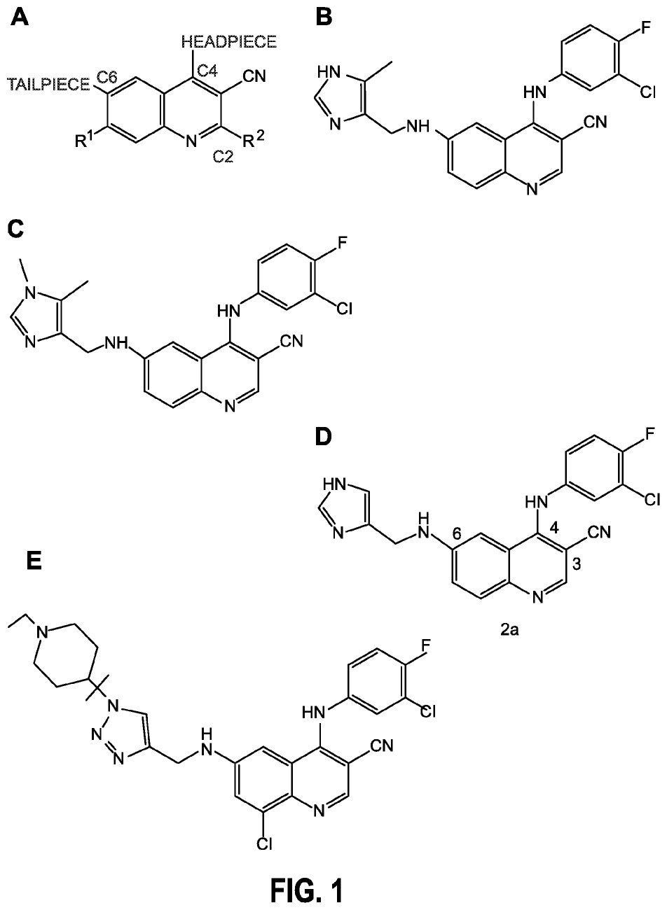

COT inhibitors can be obtained by the modification of the headpiece and tailpiece structure of carbonitriles (FIG. 1A). See: Kaila, N.; et al., Identification of a novel class of selective Tpl2 kinase inhibitors: 4-alkylamino-[1,7]-naphthyridine-3-carbonitriles. Bioorg. Med. Chem. 2007, 15 (19), 6425-6442; Hu, Y.; et al., Inhibition of Tpl2 kinase and TNFα production with quinoline-3-carbonitriles for the treatment of rheumatoid arthritis. Bioorg. Med. Chem. Lett. 2006, 16 (23), 6067-6072; and, Gavrin, L. K. et al., Inhibition of Tpl2 kinase and TNFα production with 1,7-naphthyridine-3-carbonitriles: Synthesis and structure-activity relationships. Bioorg. Med. Chem. Lett. 2005, 15 (23), 5288-5292; Wu, J.; et al., Selective inhibitors of tumor progression loci-2 (Tpl2) kinase with potent inhibition of TNF-α production in human whole blood. Bioorg. Med. Chem. Lett. 2009, 19 (13), 3485-3488, and Green, N. et al. Hotchandani, R. Inhibitors of tumor progression loci-2 (Tpl2) kinase and tumor necrosis factor α (TNF alfa) production: Selectivity and in vivo anti-inflammatory activity of novel 8-substituted-4-anilino-6-aminoquinoline-3-carbonitriles. J. Med. Chem. 2007, 50 (19), 4728-4745.

The above papers describe modification of the tailpiece into either 4-benzylphenyl or into 4-(phenylthio)phenyl to achieve a COT inhibition within the nM range. By modification of the tailpiece structure to that shown in FIG. 1B or 1C, TPL2 inhibitory effect reached an IC50 of 3 nM. The structures shown in FIG. 1, which are also shown below, represent the 4-alkylamino-[1,7]naphthyridine-3-carbonitriles class of selective Tpl2 inhibitors. Further modification of the tailpiece with an addition of a triazole as shown below (FIG. 1D) retains specificity, adds physicochemical properties, and improves potency in human blood, which enables oral administration and efficacious reduction of TNF-alfa secretion. These compounds are potent in cellular and human blood assays indicating their functional ability to inhibit COT and thus inhibit release of TNF-alfa. They also show good in vivo activity indicating good pharmacokinetic properties.

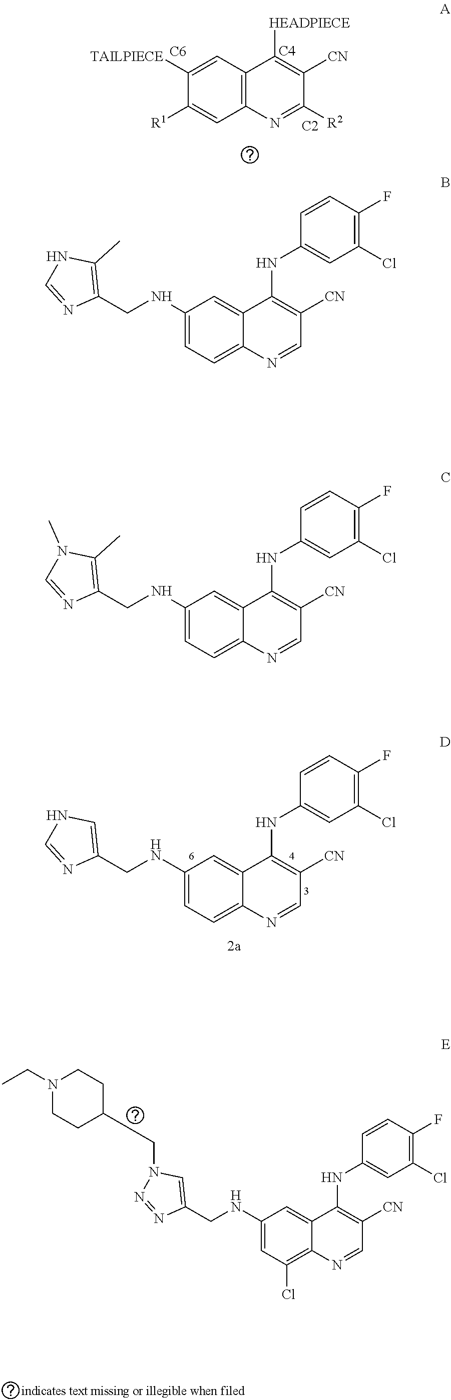

B. Indazole

COT inhibitors can be obtained by the modification of indazoles (FIG. 2). See, Hu, Y. et al. Discovery of indazoles as inhibitors of Tpl2 kinase. Bioorg. Med. Chem. Lett. 2011, 21 (16), 4758-476. This paper describes the modification of Indazoles by a benzimidazole ring (FIG. 2A) with (COT IC50 418 nm) or without a methyl group (TPL2 IC50 109 nm) on position 5 of the benzimidazole Another compound that has a modification on the C5 residue of the Indazole ring, shown below and in FIG. 2B, produced a compound that inhibits ERK phosphorylation with a COT IC50 of 47 nM. The compound in FIG. 2A, which is also shown below, with no methyl group shows good kinase selectivity against IKKβ, JNK1α, p38, EGFR, CK1α, while showing less selectivity against other kinases, including CDK2, JAK3, MST4, and GSK3β.

benzimidazole Indazole

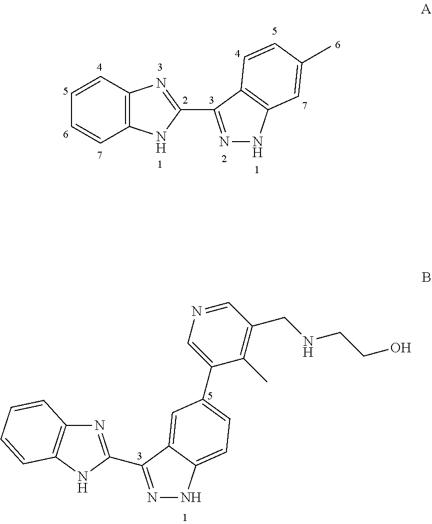 [SMILES: Cc1ccc2c(-c3nc4ccccc4[nH]3)n[nH]c2c1 ; Cc1c(CNCCO)cncc1-c1ccc2[nH]nc(-c3nc4ccccc4[nH]3)c2c1]

C. Thieno[2,3-c]pyridine and thieno-[3,2-d]pyrimidine

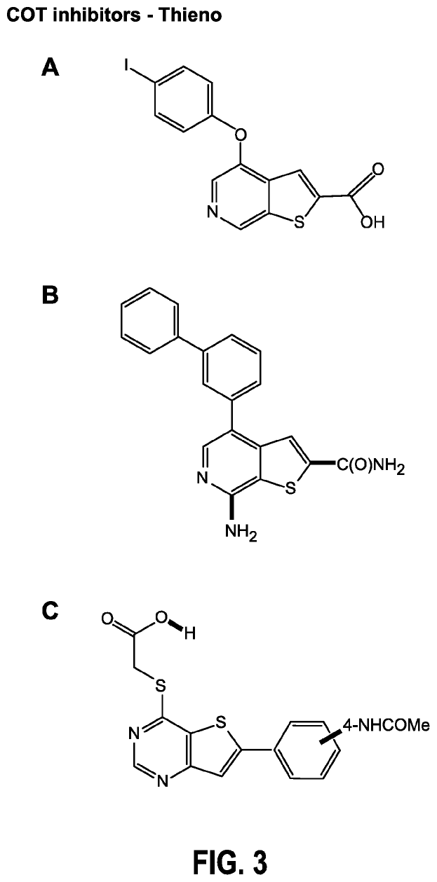

Alternative COT inhibitors can be obtained by the modification of thieno[2,3-c]pyridines as shown below and in FIG. 3. See, George, D., et al. Discovery of thieno[2,3-c]pyridines as potent COT inhibitors. Bioorg. Med. Chem. Lett. 2008, 18 (18), 4952-4955. This paper describes a series of 2,4-disubstituted thieno[2,3-c]pyridines as COT inhibitors. The compound in FIG. 3A shown below inhibited COT with an IC50 of 0.94 micromolar. A subsequent paper by some of the same scientists described additional COT inhibitors of COT. Cusack, K. et al. Identification of a selective thieno[2,3-c]pyridine inhibitor of COT kinase and TNF-α production. Bioorg. Med. Chem. Lett. 2009, 19 (6), 1722-1725. The compound in FIG. 3B where the R1 group with NH2 and the R2 group was C(O)NH2) inhibited COT with an IC50 of 0.17 micromolar, while its inhibition of four other related kinases was at least ten-fold less effective. This compound was also active when added to intact blood cells.

COT Inhibitors—Thieno

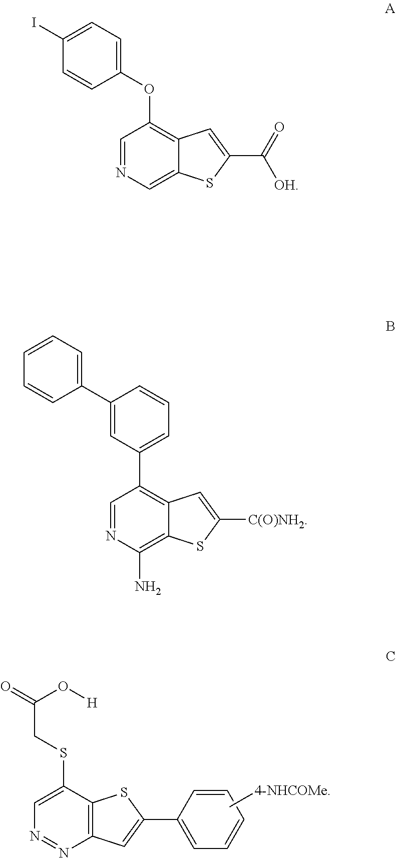

Additional COT inhibitors can be obtained by the modification of thieno[2,3-c]pyrimidines. See, Ni, Y., et al. Identification and SAR of a new series of thieno[3,2-d]pyrimidines as Tpl2 kinase inhibitors. Bioorg. Med. Chem. Lett. 2011, 21 (19), 5952-5956. The compound in FIG. 3C inhibited COT with an IC50 of 0.18 microM, had at least a 10 fold higher IC50 towards of panel of 10 other similar kinases, was active when added to intact cells, and bound to the COT protein.

Examples of cell cycle inhibitors include: Palbociclib (PD0332991), Ribociclib (LEE011), Roscovitine (Seliciclib, CYC202), Abemaciclib (LY2835219), SNS-032 (BMS-387032), Dinaciclib (SCH727965), Flavopiridol (Alvocidib), AT7519.

Examples of DNA repair inhibitors include: Veliparib, Rucaparib, Talazoparib, INO-1001, Niraparib, Pamiparib, E7449, Iniparib.

The COT inhibitory activity of existing and novel COT inhibitors can be measured using known assays. Three exemplary and useful assays for measuring COT inhibitory activity are described below.

(i) Cell-Free Kinase Assay

COT activity can be directly assayed using GST-MEK1 as a substrate as shown in Garvin, L. K., et al., Bioorg. Med. Chem. Lett. 2005, 15 (23), 5288-5292. The assay detects phosphorylation on serine residues 217 and 221 of GST-MEK1 by an ELISA. Briefly, 0.4 nM Tpl2 (COT) is incubated with 35 nM GST-MEK1 in a kinase reaction buffer with and without the test compound inhibitor. The kinase reaction is stopped with the addition of 100 mM EDTA and the reaction mix transferred to a detection plate pre-coated with an anti-GST antibody (GE Healthcare) for detection of phosphorylation.

(ii) TPL2/COT Growth Inhibition Cell-Based Assays

COT inhibitors activity will affect cell proliferation and can be monitored similar to other tyrosine and serine threonine kinase inhibitors utilizing MTT/XTT Mosmann T. J. Immunol. Meth. 1983; 65:55-63. Alamar Blue Ahmed S A, et al. J Immunol Meth. 1994; 170:211-224. In both assays, the cells are incubated with a non-fluorescent reagent that penetrates the cells, which is then metabolized to a fluorescent reagent. The amount of fluorescence is proportional to the number of viable cells in the well. In Fernandez, M., et al., Biochem Biophys Res Commun. 2011; 411(4):655-60, cells were incubated with or without (control) COT inhibitor for 30 hours, after which an MTT assay was performed. The inhibitory effect is calculated by the fluorescence obtained in the well with COT compared to the control well with no drug treatment.

(iii) In Vivo TNF Alpha Assay to Measure the Effect of COT Inhibitors

TNF α production can be used to test for COT inhibitory effect. The signal transduction pathway that stimulates TNFα expression include several members of the mitogen activated protein kinase (MAP kinase) family, including COT, a serine/threonine kinase in the MAP3K family. Therefore, activation of COT may be measured by the level of TNFα messenger RNA and TNFα protein as well as increased phosphorylation of MEK1 and ERK1 Hu, Y. et al; Inhibition of Tpl2 kinase and TNFα production with quinoline-3-carbonitriles for the treatment of rheumatoid arthritis. Bioorg. Med. Chem. Lett. 2006, 16 (23), 6067-6072. In this assay we are evaluating LPS/D-Gal-induced acute TNFα production in mouse sera: Female C57Bl/6 mice, 8-10 weeks of age, obtained from the Jackson Laboratory. Compound at 25 or 50 mg/kg, or vehicle, are administered to the mice by the intraperitoneal (ip) route. One hour after the compound, LPS plus d-galactosamine in PBS was administered ip. Final LPS and D-gal concentrations in each animal were 2 and 160 ng/kg, respectively. The mice were euthanized with carbon dioxide 1.5 h after the LPS/D-Gal injection and the mice were bled by cardiac puncture. TNFα levels were measured in the serum samples using common TNFα ELISA.

### 6. Administration and Formulation of COT and Oncogene Inhibitors

A pharmaceutical composition for treating the cancers of this invention is formulated to be compatible with its intended route of administration. Examples of routes of administration include parenteral, e.g., intravenous, intradermal, subcutaneous, oral, ocular, transbucal, nasal (e.g., inhalation), transdermal (i.e., topical), transmucosal, and rectal administration. Solutions or suspensions used any of these methods can include many additional components and/or excipients, including (but not limited to) the following: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy flow through a syringe exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active anti-cancer agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives, and/or the use of electric current. Transmucosal administration can be accomplished through the use of nasal sprays or suppositories, and/or other methods. For transdermal administration, the active compounds are formulated into ointments, salves, gels, patches, creams or other methods, as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The administration of the inhibitor of COT and the administration of the additional cancer therapeutic do not need to be at the same time or via the same route of administration. One or more inhibitors of COT may be used, or one or more additional therapeutic may be used, or both. The timing or schedule of administration of each agent may be varied in all possible combinations. Possible routes of administration for each agent may include intravenously, by injection, subdermally, intramuscularly, parenterally, intraperitoneally, orally, sublingually, transbuccally, inhalationally, nasally, rectally, transdermally or by any other method used to administer a pharmacological agent. Administration of COT inhibitor can take place as a first line drug together with another drug such as MEK1/2 inhibitor or ERK1/2 inhibitor or any PI3 kinase or mTOR or AKT inhibitor or EGFR or cMET inhibitor, or any other RTK inhibitor or any other signaling pathway inhibitor. It could also take place after initial first line treatment began but during and in parallel to that treatment. This may take place after COT expression was diagnosed to be present. Similar administration can take place together or during 2^{nd} or 3^{rd} line therapy.

A triple combination therapy is also an option. A combination of COT inhibitor together with RTK inhibitor (EGFR for example) together with MEK1/2/ERK1/2 or any PI3 Kinase inhibitor. COT Triple combination may also take place with MEK1/2/ERK1/2 inhibitors and PI3 inhibitors. The administration of the drugs may occur in the same table/capsule, as 2 tablets/capsules or via two different delivery systems such as a tablet and an injection. The drugs may be given at a different time of day like before and after a meal, morning and evening, even and odd days/weeks and so forth.

The dose of the combination of inhibitors administered to a patient, in the context of the present invention should be sufficient to effect a beneficial therapeutic response in the patient over time. The dose will be determined by the efficacy of the particular inhibitor employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the inhibitors to be administered in the treatment the physician evaluates circulating plasma levels of inhibitor, its relative toxic effects, and progression of the disease. More specifically, the administration of the many inhibitors identified herein can be administered at a rate determined by the LD-50 of the inhibitor and its the side-effects at various concentrations, as applied to the mass and overall health of the patient. Administration can be accomplished via single or divided doses. For many of the inhibitors the safe dosage window is known or readily determined using standard approaches. In general, the dose equivalent of an inhibitor or cancer cells in vivo ranges from from about 0.2 miligrams to 2 grams for a typical 70 kilogram patient per day.

The effects of combining the administration of the COT inhibitor and the additional therapeutic may significantly extend the clinical benefit of current drugs and thereby provide significant life extension and improvement of quality of life to cancer patients. This approach may be particularly useful in cancers that are hard to treat and that have few, if any, therapeutic options at the present time.

### 7. Determination of Optimal Therapeutic Response

Patient cancer treatment is calculated based on date of initial diagnostics and first line treatment or initial diagnostic of relapse/resistance to current treatment. Increase of several parameters indicate that clinical benefit response to therapy may be due to the combination of the using a variety of conventional measures and parameters.

Overall survival (OS) is a typical method and measures how long a patient lives, usually from the beginning of treatment. Progression-free survival (PFS) is an alternative measure and evaluates s how long a person lives without the disease worsening beyond a certain and/or pre-defined extent. We can assess PFS in terms of tumor progression, the appearance of new lesions, and/or death (due to any cause). An increase in PFS can mean delayed symptoms, lessened anxiety and uncertainty associated with disease progression, and increased quality of life. Disease/relapse-free survival (DFS/RFS) represents the length of time a person lives without any signs/symptoms of disease or relapse of their cancer after completion of initial treatment in early disease. This can provide an estimation of cure rates—the goal of treatment in early disease—considerably faster than OS.

The following are additional conventional criteria that can be used to measure benefits of treatment:

Response rate (RR) is the percentage of patients whose tumor is reduced by a treatment beyond a certain amount. Similar to PFS, this could mean that the person with cancer experiences fewer symptoms associated with disease progression.

Complete response (CR) A complete response seen in a person means that a tumor has completely disappeared following treatment. No signs of cancer are visible in scans or tests.

Duration of response (DoR) is the length of time that a tumor continues to respond to a treatment from first documentation of improvement to the disease worsening again.

Pathological complete response: pCR is used in early-stage disease to assess the efficacy of a treatment prior to surgery (‘neoadjuvant’), a quicker assessment than using PFS or OS. Achieving pCR means there are no cancer cells detectable at the time of surgery and, in many cases, this predicts that the disease will not relapse.

Immune-related response criteria (iRECIST): When treating solid tumors with immunotherapy, we sometimes observe unconventional response patterns, which cannot be assessed successfully using the common criteria used to evaluate treatment efficacy. While iRECIST is not an endpoint, it is an adaptation to account for the apparent increase in tumor size caused by immune system cells entering the tumor (‘pseudoprogression’).

Minimal residual disease: MRD measures the ‘depth’ of response to a treatment in blood cancers. MRD can be an early predictor of PFS, and potentially accelerate drug development in slow-growing blood cancers. MRD uses newer, highly sensitive technologies to search for traces of certain blood cancers, where traditional tests may have not detected anything.

Each of the above criteria contribute to the ability to define the clinical benefit of the patient cohort or a fraction of the cohort that possess certain common characteristics. Measuring any of the above parameters may be done by a combination of the following methodologies:

Reduction in Specific Symptom: good examples are (but not limited to), Persistent cough or blood-tinged saliva, A change in bowel habits, Blood in the stool, Unexplained anemia (low blood count), breast lump or breast discharge, lumps in the testicles, a change in urination, blood in the urine, persistent lumps or swollen glands, unexpected weight loss, night sweats, or fever.

It is common to use an imaging system (X-Ray, CT, MRI etc.) to calculate-estimate tumor size or volume followed by tumor load, shrinkage or proliferation. Blood or Body Fluid-Based Biomarkers: Biomarker presence or disappearance form the biopsy or liquid biopsy or body fluids. For example:
Decrease/increase in CA-125 is considered recovery/progression in ovarian cancer patients
Hemoglobin as a measure for anemia and further cancer indicator
Certain gene expression like a marker for relapse (example EGFR T790M)
Certain target or signaling molecule presence, phosphorylation (EGFR, ERK1/2)

### EXAMPLES

The following examples are provided by way of illustration only and not by way of limitation. Those of skill will readily recognize a variety of noncritical parameters that could be changed or modified to yield essentially similar results.

All publications and patent applications cited in this specification are herein incorporated by reference as if each individual publication or patent application were specifically and individually indicated to be incorporated by reference.

The experiments shown below reveal the capacity of combination therapy with drugs that i) have been previously approved for the indication from which the cell line has been originated, or ii) have not been approved or failed, or iii) with investigational drugs that are in clinical trials, or iv) drugs that have been shown in the literature to have potential clinical benefit. Cell lines have been obtained from the ATCC, media was obtained from Millipore Sigma, HyClone Laboratories, Life Technologies/Gibco, and ThermoFisher Scientific. Fetal bovine serum was obtained from VWR Scientific or HyClone Laboratories. siRNAs obtained from Dharmacon and from AUM Biotech, RNase free water from Dharmacon and Invitrogen, and Lipofectamine RNAiMax transfection reagent from Thermo Fisher Scientific. Drugs and chemical inhibitors were obtained from Selleckchem, AdooQ, MedChemExpress, Cayman Chemical and Abcam. All other reagents and supplements were from commercial sources.

The experiments have been performed in the following way. Three thousand cells in suspension in the medium normally used to grow these particular cells have been placed in an individual well within a 96 well tissue culture plate. Following cell attachment, the drugs have added to the wells to achieve the final indicated concentration. Subsequently, the cells were incubated for 72 hours in a standard tissue culture incubator at 37 degrees Celsius in an atmosphere containing 5% CO₂. Following 66 hours, Alamar blue was added to the cells. Six hours later (72 hours from the addition of drug) the 96 well plate was placed in a fluorescence plate reader to measure for Alamar blue fluorescence (excitation wavelength of 540-570 nm (peak excitation is 560 nm)). Fluorescence emission was read at 580-610 nm (peak emission is 590 nm). This enabled the determination of the number of live cells present in each well. Wells containing each drug were compared to control wells where no drug was added. In each experiment, each condition was performed in multiple technical repeats of between four and eight times, by using between four and eight wells that were treated identically Each and every experiment was repeated at least three times on different days. In the data shown here, a representative example of each experiment is shown. The first bar (white) shows cells incubated with no drug, the next two bars (horizontal lines) represents the cell incubated with a single tested drug followed by the COT inhibitor, both in monotherapy. The right bar (solid black) shows the effect of combining the two drugs together. Compared to monotherapy by one drug or the other, the combination of two drugs may indicate an additive or synergistic (i.e. super-additive) effect, which may translate into a superior clinical outcome.

Knockdown (KD) experiments were done as follows. The siRNA was mixed with an appropriate amount of Lipofectamine RNAiMax transfection reagent and buffers following the manufacturer's protocol for reverse transfection. The mix was placed in a 384 well ELISA Microplate from Corning and allowed to dry under sterile conditions for 48 hours. Subsequently 3,000 cells were placed in each well and incubated for 72 hours at 37° C. in a 5% CO₂ incubator and for 72 hours as described above.

### COT Inhibitor Source and Method of Use

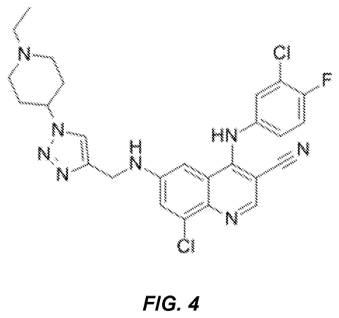

The COT inhibitor used in these experiments is 3-Quinolinecarbonitrile, 8-chloro-4-[(3-chloro-4-fluorophenyl)amino]-6-[[[1-(1-ethyl-4-piperidinyl)-1H-1,2,3-triazol-4-yl]methyl]amino]-3-quinolinecarbonitrile, CAS number 915363-56-3, with a molecular weight of 539.43. Its chemical structure is shown below as well as in FIG. 4. This inhibitor is available from several commercial sources, including BOC Sciences, MedChemExpress, ChemScene, BioCrick and others. The substance was dissolved in DMSO to a final concentration of 10 mM. Subsequent dilutions were done into aqueous solutions or in tissue culture growth media.

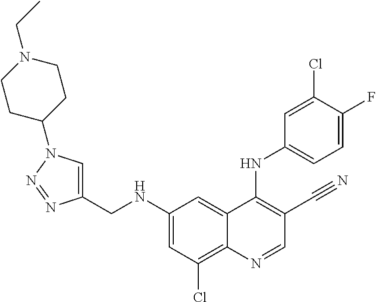 [SMILES: CCN1CCC(n2cc(CNc3cc(Cl)c4ncc(C#N)c(Nc5ccc(F)c(Cl)c5)c4c3)nn2)CC1]

### Example 1. The Effect of a Combination of a COT Inhibitor and a MEK1/2 Inhibitor (Trametinib) on Three NSCLC Cell Lines with a KRAS Oncogenic Mutation

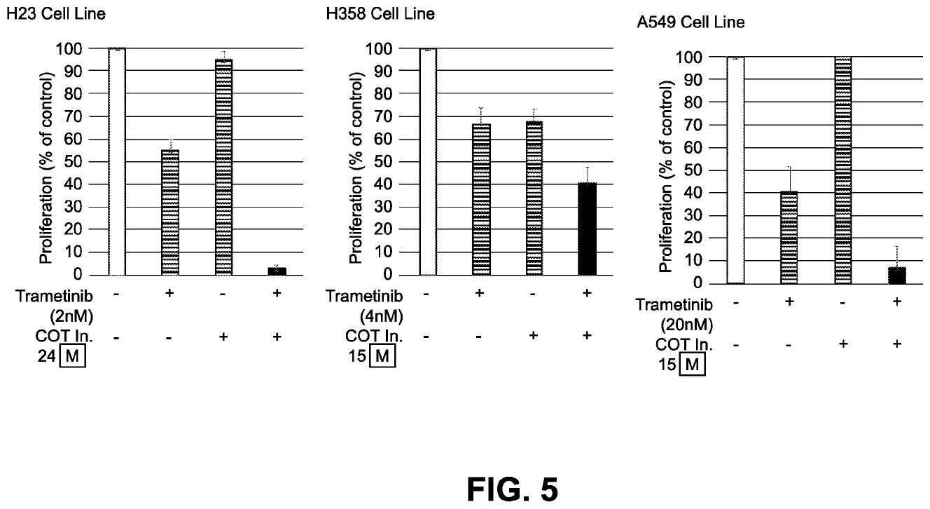

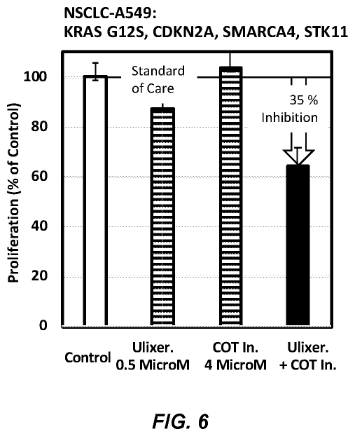

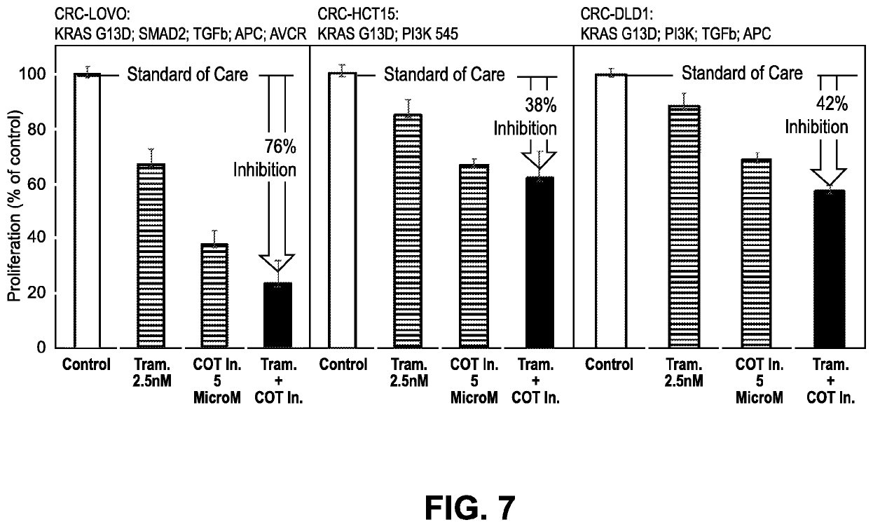

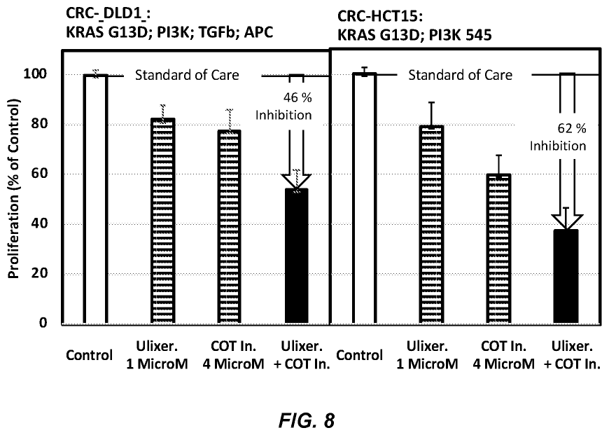

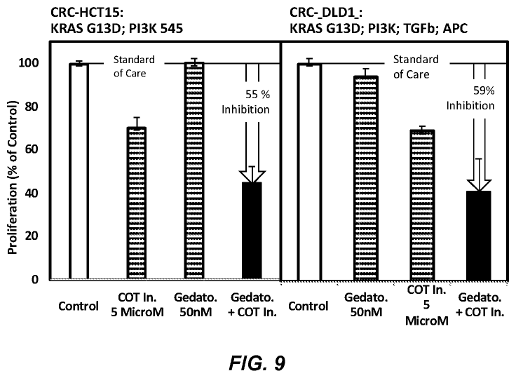

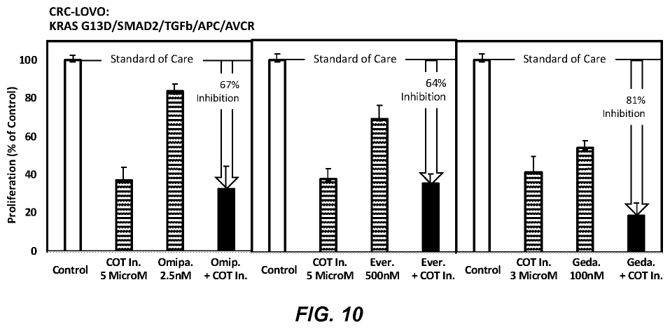

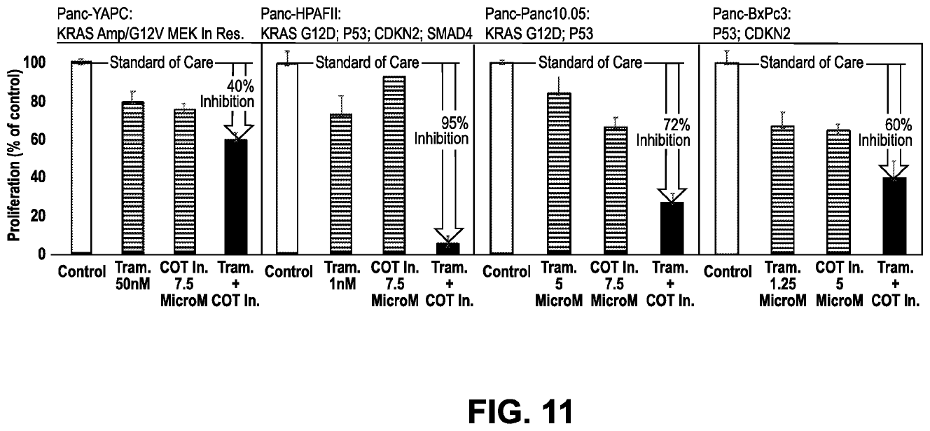

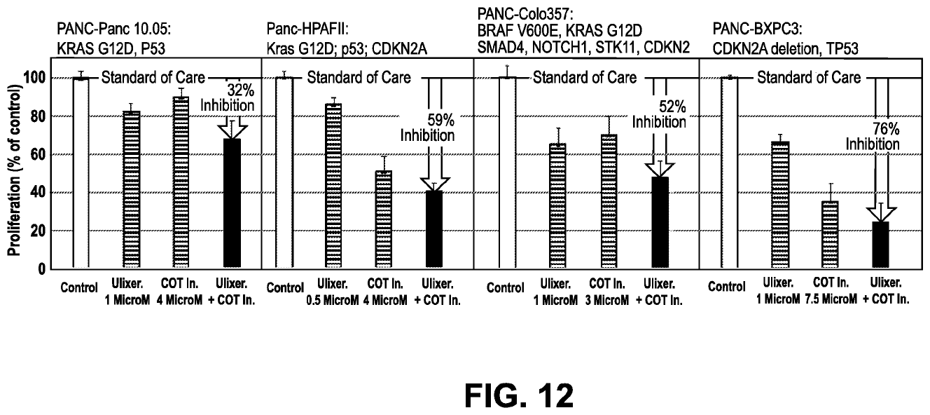

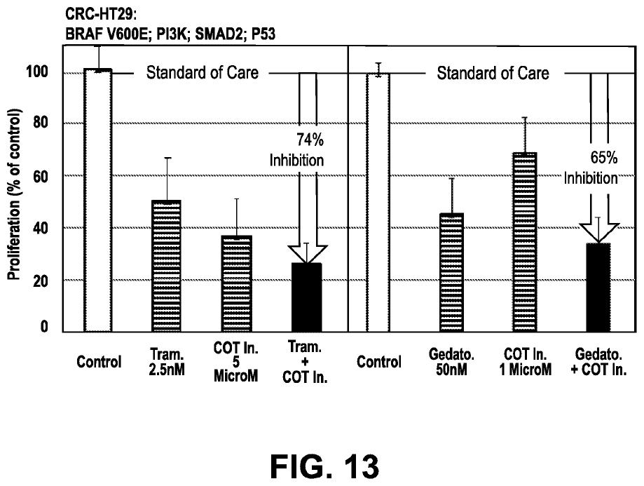

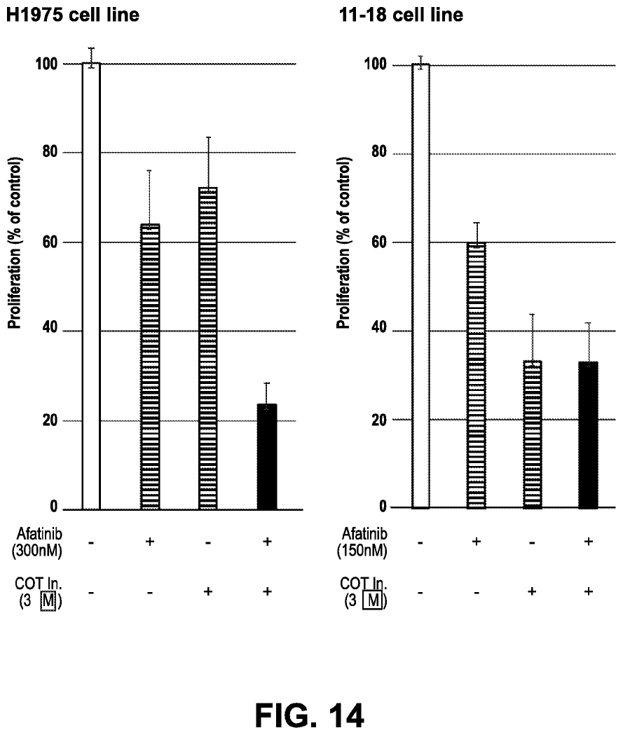

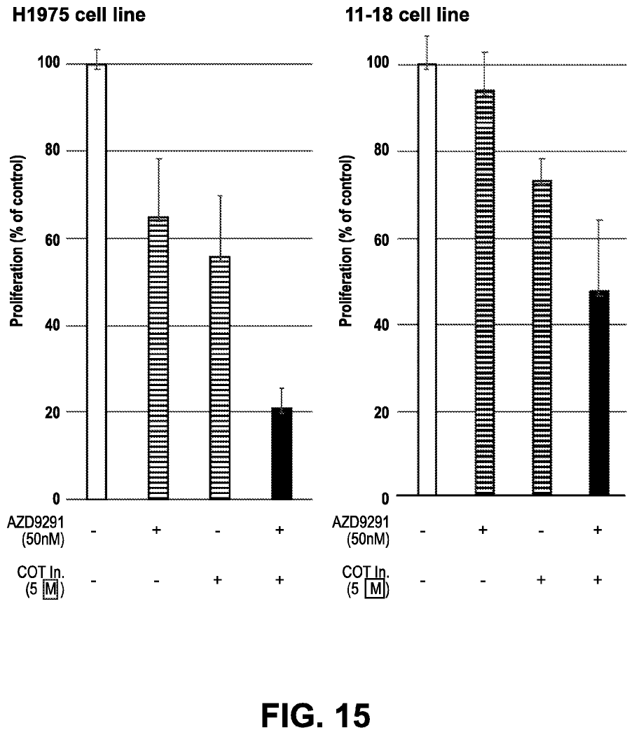

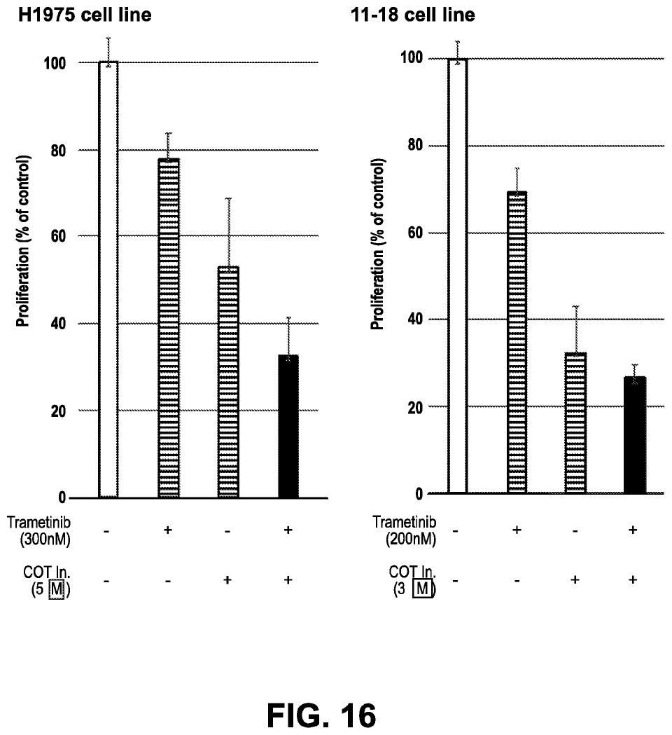

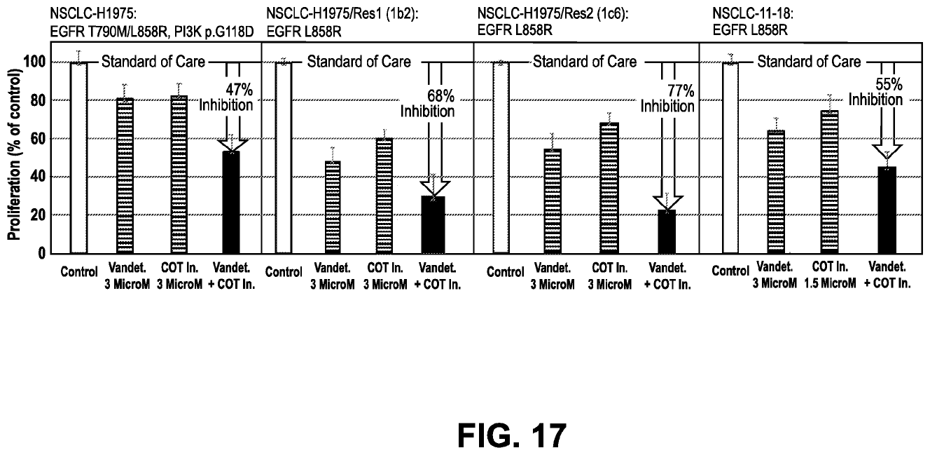

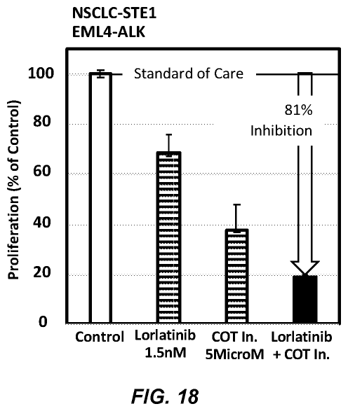

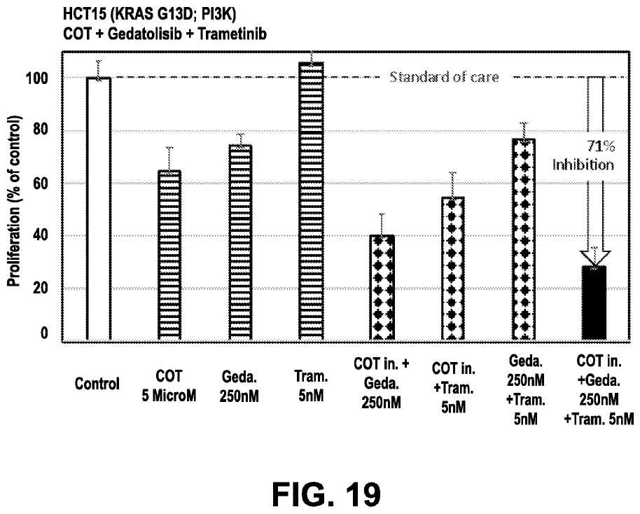

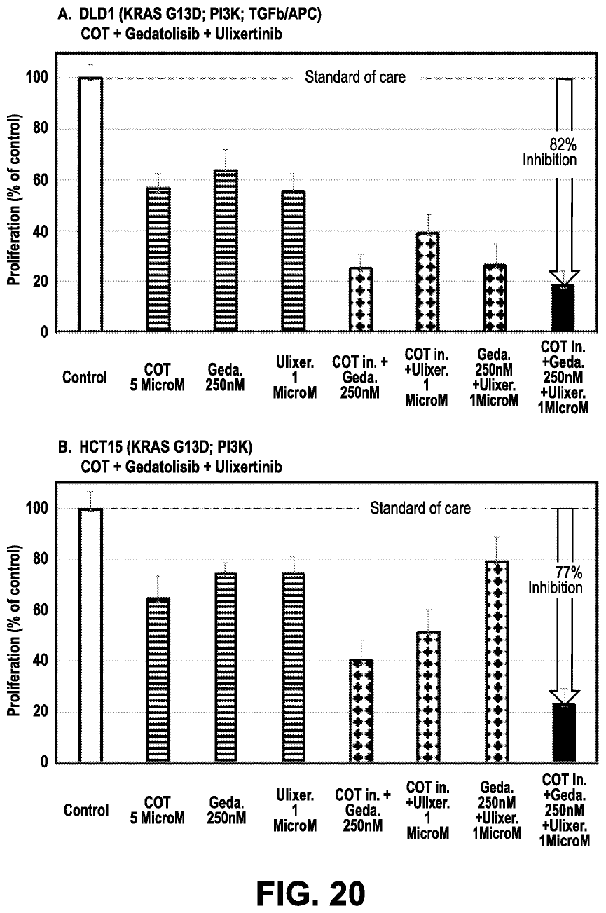

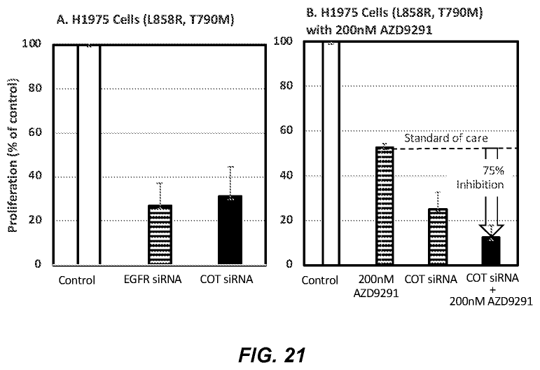

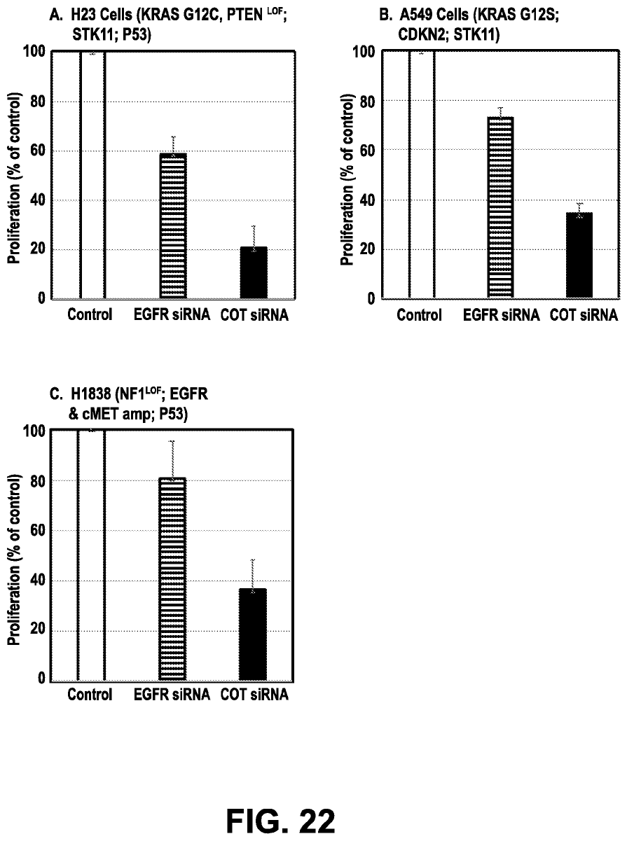

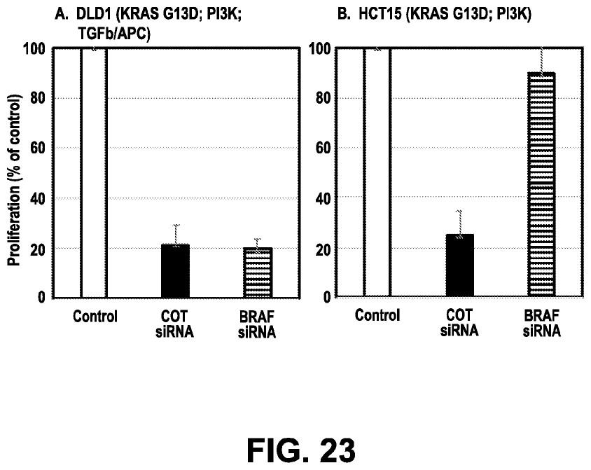

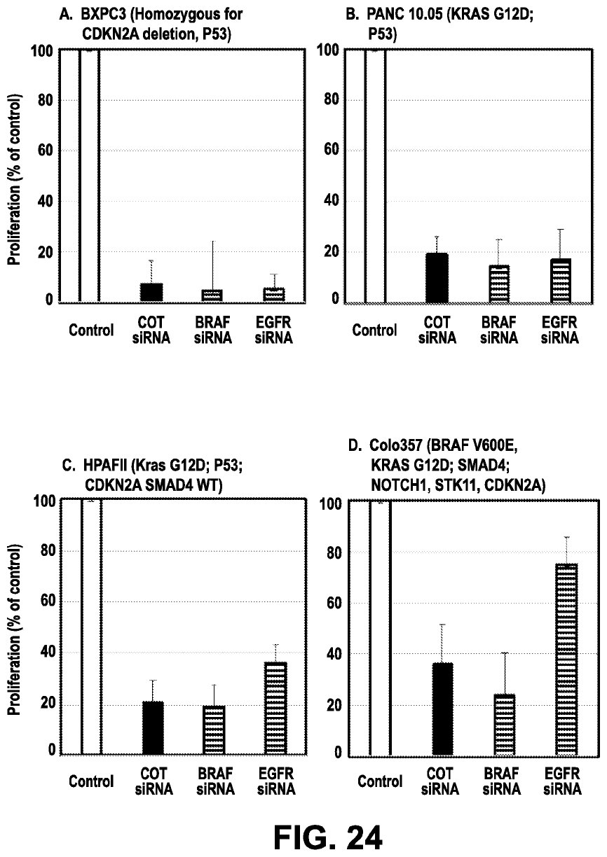

KRAS is a central signaling molecule in growth control and is mutationally activated to an oncogenic form in many NSCLC and other cancers. Currently no approved drugs target oncogenic KRAS. One approach to overcome this lack of drugs for activated KRAS is to inhibit signaling molecules that are downstream of KRAS, such as the kinases MEK1 and MEK 2 (MEK1/2, also known as dual specificity mitogen-activated protein kinase kinase 1/2). Trametinib is a MEK1/2 inhibitor that failed to show clinical benefit in NSCLC patients with KRAS mutations (NCT01362296; Dompe, N., et al., (2018). A CRISPR screen identifies MAPK7 as a target for combination with MEK inhibition in KRAS mutant NSCLC. PLOS ONE, 13(6), e0199264). In the following experiment, we demonstrate the benefit of using a combination of a COT inhibitor with a MEK1/2 inhibitor using three NSCLC cell line models: H358, H23, and A549 (FIG. 5). All three cell lines contain an oncogenic KRAS mutation. Following the general methodology provided above, we showed an additional inhibitory effect when our COT inhibitor was combined with trametinib. H23 and A549 cell lines (NCI60 cell lines) strongly support that the co-inhibition of COT provides significant clinical benefit.

### NSCLC KRAS: COT+ERK

Inhibitors of ERK1/2 (Extracellular signal-Regulated Kinases 1/2) as monotherapy or in combination with other inhibitors could provide an additional indirect strategy to inhibit oncogenic KRAS. Ulixertinib is an ERK1/2 inhibitor investigational drug used in the experiment shown below on the A549 NSCLC cell line (FIG. 6). Under the assay conditions Ulixertinib as monotherapy provides a 10% inhibitory effect. The COT inhibitor alone had no effect. The combination of the two inhibitors gave a 3.5 fold greater effect than Ulixertinib, i.e. a total of 35% inhibition. This result indicates the ability of COT inhibitor combined with ERK1/2 inhibitor to provide a much higher inhibition than ERK1/2 inhibition alone. This result supports a using a such a combination strategy in the clinic.

Direct Inhibitors of KRAS (Kirsten rat sarcoma) that inhibit specific mutants as monotherapy or in combination with other inhibitors could provide an additional strategy to inhibit oncogenic activity and generate clinical benefit. AMG510 is a KRAS G12C specific inhibitor investigational drug used in the experiments on H23 and H358 NSCLC cell lines. Under the assay conditions, AMG510 as monotherapy provides an inhibitory effect of 50% on H23 cells and 30% on H358 cells. The COT inhibitor alone had about 30% inhibition effect on both cells. The combination of the two AMG510 and COT inhibitors gave an additional inhibitory effect that accumulates to 80% on H23 cells and 55% on H358 cells. This result indicates the ability of COT inhibitor combined with KRAS direct inhibitors to provide a much higher inhibition than AMG510 alone. This result supports using such a combination strategy in the clinic.

### Example 2. Colorectal Cancer. KRAS: COT+MEK Inhibitors

KRAS is a major oncogene in colorectal cancer (CRC). If the patient's tumor harbors KRAS in the WT form, the patient will be treated with Cetuximab, an EGFR inhibiting antibody that can provide major clinical benefit. Nevertheless, 44% of the colorectal cancer patients harbor an oncogenic KRAS and have no FDA approved targeted therapy, leaving them only to chemotherapy that provides little clinical benefit.

In the following experiment (FIG. 7), three colorectal cell lines (LOVO, HCT15, DLD1) harboring an oncogenic KRAS have been used to test the potential of COT inhibition providing additional benefit in combination with the MEK1/2 inhibitor trametinib. As shown in FIG. 7, MEK1/2 inhibitor trametinib or COT inhibitor, each added alone, variable, but significant, inhibitory effect. Importantly, the combination of COT and MEK1/2 inhibitors provides a greater level of inhibition, suggesting a potential clinical benefit to patients. The results of the LOVO cell line, which does not have a PI3K mutation, provide an outstanding inhibition (76%), potentially indicating that the KRAS signaling is leaking to the COT pathway, thus generating a parallel resistance pathway. This pathway may be inhibited by a COT inhibitor, while addition of MEK1/2 inhibitor will inhibit the KRAS-BRAF-MEK-ERK pathway. Finally, this combination of MEK1/2 and COT inhibition can provide clinical benefit to the large patient cohort of colorectal patients who have a KRAS oncogenic mutation.

### Colorectal Cancer, KRAS: COT+ERK Inhibitors

The experiments below were performed on CRC cell lines and indicate the synergistic effect of combining a COT inhibitor together with the ERK1/2 inhibitor Ulixertinib. In the DLD1 CRC cell line, monotherapy with either inhibitor gave an effect of approximately 20%. The combination of the two inhibitors, however, gave more than double this, 46%. A greater effect was achieved in HCT15 cells. In this case the effect of combination of Ulixertinib tripled from 20% inhibition to 62% inhibition (FIG. 8). Both CRC cell lines showed a synergistic sensitivity to a combination of COT and ERK1/2 inhibition.

### CRC KRAS: COT+PI3K Inhibitors

Many colorectal tumors harboring oncogenic KRAS have additional oncogenic mutations. Oncogenic PI3K (phosphatidylinositol 3-kinase) is frequently associated with oncogenic KRAS, generating multiple activated signaling pathways. This scenario is difficult to treat as inhibition of one signaling pathway often results in hyper-activation of the other signaling pathway. We examined HCT15 and DLD1 CRC cell lines. In FIG. 9, incubation of these two cell lines with Gedatolisib, which inhibits both relevant PI3 kinases (PI3Kα, PI3Kγ) and mTOR, shows little to no inhibition when used alone. When COT inhibitor is incubated alone it provides a 30% inhibition. This may be attributed to the activation of the COT pathway by both KRAS and PI3K. When Gedatolisib is combined with COT inhibitor, a much greater inhibitory effect is observed (55% and 59%). This suggests that such a combination is likely to provide a higher benefit to patients in the clinic.

The Effect of the Family of PI3K/mTOR (Mammalian Target of Rapamycin) Inhibitors on the CRC LOVO Cell Line that Harbors Oncogenic KRAS

To test the effect of the family of PI3K/mTOR (mammalian Target of Rapamycin) inhibitors on the CRC LOVO cell line that harbors oncogenic KRAS, but is devoid of oncogenic PI3K, we have incubated this cell line with 3 different investigational or FDA approved drugs that inhibit the PI3K signaling pathway. These include Everolimus (mTOR inhibitor), Omipalisib (mTOR/PI3K inhibitor) and Gedatolisib (PI3Kα, PI3Kγ and mTOR inhibitor). FIG. 10, shows that when used alone, the PI3K/mTOR inhibitors provide 15 to 45% inhibition. The incubation of the COT inhibitor alone causes a 50 to 60% inhibition. When the PI3K/mTOR inhibitors are combined with the COT inhibitor, the Gedatolisib provides an advantage resulting from the combination, generating an 81% inhibition. In the cases of Everolimus and Omipalisib the combined inhibitory effects of 64% and 67% inhibition, respectively, which is equal to the effect of COT inhibitor alone. The result support the use of COT in monotherapy in certain cases, as well as in combination therapy for oncogenic KRAS bearing CRC tumors to provide a better clinical benefit.

### Example 3: Pancreatic Cancer KRAS

### Pancreatic Cancer KRAS: COT+MEK Inhibitors

95% of non-endocrine pancreatic cancer patients carry oncogenic KRAS that is believed to be the prime source for oncogenicity in such cancers. To date, no targeted therapy drug has been approved for the direct or indirect inhibition of oncogenic KRAS in pancreatic cancer. In the experiments shown in FIG. 11 either MEK1/2 inhibitor or COT inhibitor when incubated with the cells alone produced limited inhibitory effect limited to 30% maximal inhibition. Nevertheless, when they have been combined together, they produced a synergistic and significant inhibitory effect ranging from 40% to 95%, far exceeding the best monotherapy result. This indicates that while each inhibits an active signaling pathway in these cells, there is an additional, parallel signaling pathway that compromises the inhibitory effect of each drug. When both are inhibited (MEK1/2 and COT together), there is a large inhibitory effect indicating that the major pathways active in these cells are inhibited. Altogether, the results demonstrate that this combination can provide clinical utility for patients that currently have no approved treatment.

### Pancreatic Cancer KRAS: COT+ERK Inhibitors

The ERK1/2 inhibitor Ulixertinib has been considered for treatment of pancreatic cancer patients (NCT02608229). We have tested its effect alone and in combination with COT inhibitor on four pancreatic cancer cell lines. All cell lines when treated with Ulixertinib showed variable results of inhibition ranging from 15% to 35% inhibition. Combination of Ulixertinib with COT inhibitor provided synergistic results. In BxPC3 it doubled the inhibitory effect from 32% inhibition to 76% and in HPAFII it quadrupled the inhibitory effect from 15% to 59%. The results signify that Ulixertinib and COT inhibitor will have clinical benefit in pancreatic cancer.

### Example 4. The Effect of a Combination of COT Inhibitor and MEK1/2 Inhibitor (Trametinib) on patients with a BRAF Oncogenic Mutation

8-15% of CRC tumors harbor BRAF mutations (De Roock W. et al., Clinical biomarkers in oncology: focus on colorectal cancer, Mol Diagn Ther. 2009; 13(2):103-14, Rizzo S., et al., Prognostic vs predictive molecular biomarkers in colorectal cancer: is KRAS and BRAF wild type status required for anti-EGFR therapy? Cancer Treat Rev. 2010 November; 36 Suppl 3:S56-61; Tejpar S. et al., Prognostic and predictive biomarkers in resected colon cancer: current status and future perspectives for integrating genomics into biomarker discovery. Oncologist. 2010; 15(4):390-404) and this is associated with decreased overall survival (Roth A. D., et. Al., Prognostic role of KRAS and BRAF in stage II and III resected colon cancer: results of the translational study on the PETACC-3, EORTC 40993, SAKK 60-00 trial, J Clin Oncol. 2010 Jan. 20; 28(3):466-74). Additionally, metastatic CRC that harbor BRAF mutations do not respond to anti-EGFR therapy even in the presence of WT KRAS (Mao C., et al. BRAF V600E mutation and resistance to anti-EGFR monoclonal antibodies in patients with metastatic colorectal cancer: a meta-analysis. Mol Biol Rep. 2011 April; 38(4):2219-23). The most frequently reported BRAF mutation is V600E (Rizzo S., et al., Prognostic vs predictive molecular biomarkers in colorectal cancer: is KRAS and BRAF wild type status required for anti-EGFR therapy? Cancer Treat Rev. 2010 November; 36 Suppl 3:S56-61). While BRAF V600-mutated melanomas are sensitive to vemurafenib (Sosman, J. A., et al. (2012). Survival in BRAF V600—Mutant Advanced Melanoma Treated with Vemurafenib. New England Journal of Medicine, 366(8), 707-714), BRAF V600-mutated CRCs may not be as sensitive (Prahallad, A., et al. (2012). Unresponsiveness of colon cancer to BRAF(V600E) inhibition through feedback activation of EGFR. Nature, 483(7388)).

Activation of EGFR in colorectal cancer could explain why colorectal cancers generally have a lower response to BRAF inhibitors (Corcoran, R. B., et al. (2012). EGFR-Mediated Reactivation of MAPK Signaling Contributes to Insensitivity of BRAF—Mutant Colorectal Cancers to RAF Inhibition with Vemurafenib. Cancer Discovery, 2(3), 227-235). See, king an indirect therapy approach may support the treatment of mCRC patients that harbor oncogenic BRAF. The CRC cell line HT29 that harbors oncogenic BRAF was tested for two indirect inhibition strategies. In the first strategy, COT inhibitor was combined with MEK1/2 inhibitor trametinib. In the second strategy COT inhibitor was combined with PI3K inhibitor. In both cases, each of the monotherapy approaches provided a significant inhibitory effect as well as additional inhibitory effect when combined with COT inhibitor and reached inhibitory effect of 65% to 74% inhibition. The results shown in FIG. 13 suggest an additional signaling pathway that is stimulated by BRAF and other oncogenes active in HT29 cells that can be inhibited by COT inhibitor.

### Example 5. Non-Small Cell Lung Cancer (NSCLC)

The Effect of a Combination with COT Inhibitor on Patients with Egfr Harboring an Oncogenic Mutation.

In NSCLC, 10-15% of patients in the US and 35%-50% in East Asia have tumors harboring EGFR mutations (Paez, J. G., et al. (2004). EGFR mutations in lung cancer: correlation with clinical response to gefitinib therapy. Science (New York, N.Y), 304(5676), 1497-1500; Lynch, T. J., et al. (2004). Activating mutations in the epidermal growth factor receptor underlying responsiveness of non-small-cell lung cancer to gefitinib. The New England Journal of Medicine, 350(21), 2129-2139; Pao, W., et al., (2004). EGF receptor gene mutations are common in lung cancers from “never smokers”; and are associated with sensitivity of tumors to gefitinib and erlotinib. Proceedings of the National Academy of Sciences of the United States of America, 101(36), 13306-13311). Since their discovery, three generations of EGFR inhibitor targeted therapy drugs have been successfully developed to inhibit proliferation of tumors harboring these mutations as well as the resulting resistant mutations in the EGFR, such as EGFR T790M. This effort has extended the progression-free survival from a few months to an average of 18 months when treated with osimertinib or initially with erlotinib/gefitinib/afatinib followed by osimertinib. Eventually, most patients on osimertinib develop resistance to all targeted therapies, relapse and, following a short period of conventional chemotherapy treatment, die.

### NSCLC EGFR: COT+Afatinib

The results shown in FIGS. 14 to 16 reveal that the combination of COT inhibitor with several different drugs have the potential of extending life for this patient cohort beyond the 18 months achieved by Osimertinib. Both the H1975 and 11-18 NSCLC cell lines harbor the EGFR oncogenic mutation L858R. H1975 cells harbor the resistance mutation EGFR T790M that confers resistance to the first and second generations of EGFR inhibitors (erlotinib/gefitinib/afatinib). Therefore, the H1975 cell line resembles tumor patient at early treatment phase that well responds to Osimertinib. The 11-18 cell line has acquired additional resistant gene aberrations that are osimertinib resistant. They therefore are a good model for tumors of patients at a later phase that respond less to osimertinib due to emergence of resistance. FIG. 14 shows that H1975 cells treated with EGFR inhibitor afatinib, combined with COT inhibitor, provides double the inhibitory effect compared to the cells treated with afatinib alone (35% to 75% inhibition). This indicates the potential clinical benefit from combining COT inhibitor with afatinib for NSCLC patients with oncogenic EGFR. The 11-18 cell line is a model of patients that have been treated with osimertinib and acquired resistance to osimertinib. In these cells the combined inhibition of afatinib with COT inhibitor provides 70% inhibition, which is about 30% additional inhibition and similar to COT inhibition alone. This suggests a significant potential benefit to patients treated with such a combination.

### NSCLC EGFR: COT Inhibitor+Osimertinib

Osimertinib is a third generation EGFR inhibitor that inhibits EGFR oncogenic mutations as well as overcoming the T790M resistance mutation. This drug has been approved as second line therapy for NSLC patients that were treated with first- or second-generation EGFR inhibitors and have developed a T790M resistance mutation. The drug was further approved as a first line treatment for NSCLC patients harboring an EGFR oncogenic mutation. Following either treatment, patients generally develop resistance to osimertinib and relapse. In FIG. 15 we show that in H1975 cells, the combination of osimertinib with COT inhibitor doubles the inhibitory effect compared to osimertinib alone. In the 11-18 cell line that models a late stage tumor that is already resistant to osimertinib, the combination with COT provides a 10-fold greater inhibition compared to osimertinib alone. Importantly, in these cells most of the oncogenic signaling is COT dependent, rather than EGFR dependent.

### NSCLC EGFR: COT+Trametinib

NSCLC patients that have been diagnosed originally with oncogenic EGFR and have been treated initially with erlotinib/gefitinib/afatinib, and subsequently with osimertinib, or were treated initially with osimertinib as first line, will eventually develop resistance and relapse. This resistance may be on-target resistance (i.e. a mutation within the gene encoding EGFR) (Thress K. S., et al. Acquired EGFR C797S mutation mediates resistance to AZD9291 in non-small cell lung cancer harboring EGFR 1790M. Nat Med. 2015 June; 21(6):560-2) or off-target resistance (i.e. in a gene other than that encoding EGFR) (Ku B. M. et al. Acquired resistance to AZD9291 as an upfront treatment is dependent on ERK signaling in a preclinical model. PLoS One. 2018 Apr. 11; 13(4):e0194730; Planchard D., et al., EGFR-independent mechanisms of acquired resistance to AZD9291 in EGFR T790M-positive NSCLC patients. Ann Oncol. 2015 October; 26(10):2073-8). For both on-target and off-target resistance mechanisms, there is no approved treatment. The inhibition of these resistant mechanisms may be performed by continuous monitoring of mutations and/or amplifications that may arise, followed by inhibiting them with specific inhibitors to any oncogenes that may be identified. This strategy requires high investment in repeated liquid biopsies and NGS sequencing. It also suffers from our lack of knowledge of many of the novel resistance mechanisms. Another strategy would be by inhibition of signaling proteins positioned downstream in the signaling pathways.

By inhibiting two or three such pathways, we can effectively inhibit many of the oncogenes that potentially may participate in parallel signaling and trigger resistance. COT is positioned at a unique junction of many signaling pathways. As such, by inhibiting COT we may inhibit a large number of oncogenes originating at the plasma membrane (RTKs: EGFR, FGFR, EML4-ALK, cMET, etc.) or close to it (KRAS, BRAF, PI3K, PTEN, etc.) as well as several signaling entry points to the nucleus (ERK1/2, NFkB, P38, STATs, RELA, JNK, ERK5, etc.). In FIG. 16 we show that in the two NSCLC-EGFR cell lines, downstream inhibition of both MEK1/2 (inhibiting the ERK1/2 path) and COT, provides 70% inhibition that could not be obtained by MEK1/2 inhibition alone. These results suggest that most of the resistance signaling to MEK inhibitor is in parallel pathways that are COT dependent. This strategy may be of great advantage when knowledge of tumor resistance mechanisms is not known or clear.

### NSCLC EGFR: COT Inhibitor+Vandetanib

Vascular Endothelial Growth factor receptor (VEGFR) is expressed in endothelial and cancer cells. In cancer cells, VEGFR's activation by VEGF through autocrine and paracrine pathways results in proliferation and inhibition of apoptosis (Dai J., et al. Cabozantinib inhibits prostate cancer growth and prevents tumor-induced bone lesions. Clin Cancer Res. 2014 Feb. 1; 20(3):617-30; Current Cancer Drug Targets, 2016, 16 The novel VEGF121-VEGF165 fusion attenuates angiogenesis and drug resistance via targeting VEGFR2-HIF-1α-VEGF165/Lon signaling through PI3K-AKT-mTOR pathway). Its inhibition can help tumor eradication by both inhibition of angiogenesis as well inhibition of proliferation and promoting apoptosis. In the FIG. 17 below (left panel) we show that in NSCLC H1975 cells either the VEGFR inhibitor vandetanib or COT inhibitor alone each provide 20% inhibition. The combination of the two inhibitors more than doubles the inhibitory effect to reach 47%. NSCLC 11-18 cells (right panel) that model a more progressed tumor with additional active resistance mechanisms, show a higher level of inhibition for either inhibitor alone and a 55% inhibition was reached when the two inhibitors were combined. H1975/Res1 and Res2 are two resistant clones derived from ENU mutated H1975 cells. In these cells, the effects of either the VEGFR inhibitor vandetanib or COT inhibitor alone provide higher level of inhibitory effect compared to parental cells H1975 (20% inhibition in H1975 compared to 35% to 50% in the resistant clones). When these drugs are combined, the inhibitory effect grows from 47% in H1975 to 68% in Res1 and 77% in Res2. Altogether these results suggest an increase in acquired resistance mechanisms by activated VEGFR that activates several downstream signaling pathways. The co-inhibition of VEGFR and downstream COT pathways, which provide a significantly higher level of inhibition that can provide additional clinical benefit to patients that have resisted osimertinib treatment.

### Example 6. The Effect of a Combination of COT Inhibitor and a Brigatinib/Lorlatinib (Fusion Inhibitors) on Patients with an ALK/ROS/RET Oncogenic Fusions

Several oncogenic fusions have been identified in lung cancer. These include ALK, ROS and RET fusions. The most frequent fusion is EML4-ALK: approximately 3-7% of NSCLC tumors harbor ALK fusions (Koivunen, J. P. et al. EML4-ALK Fusion Gene and Efficacy of an ALK Kinase Inhibitor in Lung Cancer. Clin. Cancer Res. 14, 4275-4283 (2008); Kwak, E. L. et al. Anaplastic Lymphoma Kinase Inhibition in Non-Small-Cell Lung Cancer. N. Engl. J. Med. 363, 1693-1703 (2010); Shinmura, K. et al. EML4-ALK fusion transcripts, but no NPM-, TPM3-, CLTC-, ATIC-, or TFG-ALK fusion transcripts, in non-small cell lung carcinomas. Lung Cancer 61, 163-169 (2008); Soda, M. et al. Identification of the transforming EML4-ALK fusion gene in non-small-cell lung cancer. Nature 448, 561-566 (2007); Takeuchi, K. et al. Multiplex Reverse Transcription-PCR Screening for EML4-ALK Fusion Transcripts. Clin. Cancer Res. 14, 6618-6624 (2008); Wong, D. W.-S. et al. The EML4-ALK fusion gene is involved in various histologic types of lung cancers from nonsmokers with wild-type EGFR and KRAS. Cancer 115, 1723-1733 (2009)). Clinically, the presence of EML4-ALK fusions is associated with EGFR tyrosine kinase inhibitor (TM) resistance (Shaw, A. T. et al. Clinical Features and Outcome of Patients With Non-Small-Cell Lung Cancer Who Harbor EML4-ALK. J. Clin. Oncol. 27, 4247-4253 (2009)).

Multiple different ALK rearrangements have been described in NSCLC. At least nine different EML4-ALK fusion variants have been identified in NSCLC (Choi, Y. L. et al. Identification of Novel Isoforms of the EML4-ALK Transforming Gene in Non-Small Cell Lung Cancer. Cancer Res. 68, 4971-4976 (2008); Horn, L. & Pao, W. EML4-ALK: Honing In on a New Target in Non-Small-Cell Lung Cancer. J. Clin. Oncol. 27, 4232-4235 (2009); Koivunen, J. P. et al. EML4-ALK Fusion Gene and Efficacy of an ALK Kinase Inhibitor in Lung Cancer. Clin. Cancer Res. 14, 4275-4283 (2008); Soda, M. et al. Identification of the transforming EML4-ALK fusion gene in non-small-cell lung cancer. Nature 448, 561-566 (2007); Takeuchi, K. et al. Multiplex Reverse Transcription-PCR Screening for EML4-ALK Fusion Transcripts. Clin. Cancer Res. 14, 6618-6624 (2008); Wong, D. W.-S. et al. The EML4-ALK fusion gene is involved in various histologic types of lung cancers from nonsmokers with wild-type EGFR and KRAS. Cancer 115, 1723-1733 (2009)). In addition, non-EML4 fusion partners have also been identified, including KIF5B-ALK (Takeuchi et al. 2009) and TFG-ALK (Rikova et al. 2007). In the vast majority of cases, ALK rearrangements are non-overlapping with other oncogenic mutations found in NSCLC (such as EGFR mutations, KRAS mutations, etc.; Inamura, K. et al. EML4-ALK lung cancers are characterized by rare other mutations, a TTF-1 cell lineage, an acinar histology and young onset. Mod. Pathol. 22, 508-515 (2009)).

Several generations of drugs have been developed and approved for ALK fusions, including crizotinib, brigatinib, alectinib, ceritinib and loraltinib. ROS1 fusions account for approximately 2% of NSCLC lung tumors (Bergethon, K. et al. ROS1 Rearrangements Define a Unique Molecular Class of Lung Cancers. J. Clin. Oncol. 30, 863-870 (2012)). Similar to ALK fusions, ROS1 fusions respond well and are approved for treatment with targeted therapy with Crizotinib, brigatinib and loraltinib. RET fusion accounts for ˜1-2% of NSCLC cases (Takeuchi, K. et al. RET, ROS1 and ALK fusions in lung cancer. Nat. Med. 18, 378-381 (2012); Kohno, T. et al. KIF5B-RET fusions in lung adenocarcinoma. Nat. Med. 18, 375-377 (2012)). Cabozantinib, vandetanib, lenvatinib, and RXDX-105 have been studied and approved for treatment of RET fusions. Cabozantinib and vandetanib have been the most heavily studied multi-kinase inhibitors (MKI), with response rates of 20% to 50% in largely pretreated patients with RET-fusions in NSCLC.

In FIG. 18 we show that the STE1 NSCLC cell line when treated with loraltinib results in 81% inhibition.

### Example 7. The Effect of a Triple Combination of COT Inhibitor and PI3 Kinase Inhibitor with MEK/ERK Inhibitors Together on Patients Harboring a KRAS Oncogenic Mutation

A significant cohort of colorectal cancer patients harboring oncogenic KRAS also harbor oncogenic PI3 kinase. Such oncogenic cancers are extremely hard to treat effectively. The colorectal cell lines HCT15 and DLD1 are good models of such cancers. Previously in FIGS. 5-17 we have shown that dual combination of COT inhibitor with either MEK, ERK, PI3K, EGFR, EML4-ALK, VEGFR inhibitors provide significant additional proliferation inhibitory effect compared to treatment with each drug alone. In the following FIGS. 19 and 20, we show that a triple combination of COT inhibitor with the PI3K/mTOR inhibitor gedatolisib and the MEK inhibitor trametinib (FIG. 18), or the COT inhibitor with the PI3K/mTOR Gedatolisib and the ERK inhibitor Ulixertinib (FIG. 19), provide a much higher proliferation inhibitory effect compared to the dual combinations or treatment with each drug alone. These results come on the background of the failures of clinical trials (NCT01390818, NCT01658943, NCT01021748, NCT01333475, NCT01378377) that tested the combination of MEK inhibitors combined with PI3K inhibitors for the treatment of tumors that express oncogenic KRAS. The results below clearly indicate on the clinical benefit of the above triple combinations in treating patients harboring oncogenic RTK, KRAS BRAF PI3K EML4-ALK and other oncogenes.

### Example 8. The Effect of COT Knockdown on Proliferation Clinical Use of Knockdown (KD) and Knockout (KO) Technologies

Knockdown and related knockout technologies are being developed to treat patients. Indeed, the FDA approved the first RNAi drug, Onpattro, in August, 2018, for treatment of hereditary ATTR amyloidosis, also known as familial transthyretin-associated amyloidosis, a rare genetic disease. In this disease, mutations in the TTR gene lead to a pathological accumulation of the related protein. Cancer patients will be treated with KD and KO technologies to inhibit oncogenic signaling. In the examples below we show that cancer patients will obtain clinical benefit from knockdown of COT expression by COT siRNA, which represents both the knockdown and the knockout (CRISPR) fields. The results shown below that either of COT KD/KO alone or the combination of knockdown of COT plus an appropriate targeted therapeutic in cell lines representing NSCLC, pancreatic cancer and CRC all show dramatic inhibition of proliferation. This indicates that COT is a viable target for knockdown and knockout cancer patient treatment.

### KD of COT in NSCLC Cells Harboring Oncogenic EGFR.

In FIG. 21, we used the NSCLC H1975 cell line that expresses EGFR containing both an oncogenic mutation (L858R) as well as a second mutation (T790M) that renders the EGFR resistant to first generation EGFR inhibitors (erlotinib/gefitinib/afatinib). The cell line represents patients that have been treated with either first or second line of EGFR inhibitors, developed resistance to such EGFR inhibitors, and then relapsed. Fifty percent of these patients respond to Osimertinib (AZD9291), which overcomes the EGFR T790M resistance mutation and is the current standard of care. In FIG. 21A, KD of EGFR and COT provides a similar effective result. In FIG. 21B incubation of the cells with 200 nM AZD9291 reduces relative cell number by ˜50% in our proliferation assay and represents the treatment benefit obtained. KD of COT shows a bigger inhibitory effect compared to 200 mM of Osimertinib. Strikingly, the combination of drug treatment (Osimertinib) together with KD of COT provides significant added inhibition (75% inhibition) compared to drug treatment alone. These results show the potential clinical benefit of inhibition of COT by KD technology in combination with a targeted therapeutic.

### Knockdown of COT on NSCLC Cells Containing Oncogenic KRAS

Perhaps the most insidious of all oncogenes are oncogenic KRAS mutations. Not only are these among the most common oncogenic mutations, but no targeted therapy has been approved, despite massive efforts. Conventional chemo- and radio-therapies provide only a brief extension of life.

H23 and A549 cells in FIG. 22A, B are both NCI60 NSCLC lines and have oncogenic KRAS G12 mutations. H23 additionally has a loss of function (LOF) of PTEN, resulting in activation of the PI3K-AKT-mTOR pathway. A549 cells have a loss of the CDKN2, resulting in loss of inhibition of the cell cycle. In both cell lines, siRNA KD of EGFR, which lies upstream of KRAS, causes some inhibition of proliferation. Importantly, KD of COT causes a much greater inhibition of proliferation, in the range of 70-80%. This is a singular achievement for a cancer cell line driven by activated KRAS.

H1838 NSCLC cells in FIG. 22C have a LOF of NF1 that upregulates the RAS-MAPK pathway, mimicking the effect of oncogenic KRAS. Here again, KD of COT inhibits proliferation by ˜65%. Results indicate the potential clinical benefit from COT KD to NSCLC patients harboring oncogenic KRAS or hyper active MAPK pathway (NF1^{LOF}).

### Colorectal Cancer Cell Proliferation Inhibited by COT KD

Forty five percent of colorectal cancer patients harbor a KRAS oncogenic mutation rendering them untreatable by effective targeted drugs. These patients are mainly treated with conventional chemotherapy drugs, relapse early and die. KD technology will have a dramatic effect on the survival of this patient cohort. In FIG. 23, we show that colorectal cancer cell model harboring a deadly combination of both oncogenic KRAS as well as a PI3 kinase mutation, respond to COT siRNA KD. In DLD1 cells treated with either COT or BRAF siRNA, cell proliferation was inhibited by 80%. In HCT15, only COT KD had an effect, while BRAF had a non-significant effect.

### Pancreatic Cancer Cells Proliferation Inhibited by COT KD

In the case of pancreatic cancers, 90-95% harbor oncogenic KRAS, with no targeted drug to overcome its oncogenic activity. This leaves conventional chemotherapy to be the mainstream drug treatment for this disease. In FIG. 24, we show that in four cell line models, KD of COT provides a similar effect to BRAF KD and in some cases a significantly better effect than KD of EGFR. BxPC3 cells, which are devoid of KRAS oncogenic mutation, show greater than 90% proliferation inhibition by either COT, BRAF or EGFR. PANC 10.05, which harbors oncogenic KRAS G12D, shows an 80% proliferation inhibition by KD of any of the 3 constructs. HPAFII, which on top of oncogenic KRAS, harbors oncogenic SDKN2, also responds to COT and BRAF with 80% proliferation inhibition, though it responds less to EGFR KD (65%). Colo357 harboring both BRAF and KRAS oncogenic mutations as well as a set of 4 additional oncogenic mutations. This cell lines shows little inhibitory effect from EGFR KD, but significant inhibitory effect from BRAF and COT KD (76% and 65% respectively). In pancreatic cancer cell models, COT inhibition provides better inhibitory effect than EGFR KD, and a similar effect to BRAF KD. This puts COT as a first line of genes whose KD can provide clinical benefit for pancreatic cancer.

### Summary of Examples

The examples provided above show that for numerous indications and oncogenes, combining a wide variety of targeted therapeutics with COT inhibitor provides a significantly greater inhibitory effect than using the targeted therapeutic alone. In this study, we have included cell lines from three different indications (NSCLC, CRC and pancreatic cancer), as well as several different groups of oncogenes and different classes of inhibitors. The oncogenes included RTKs such as EGFR and VEGFR, cytoplasmic signaling proteins such as KRAS and BRAF, PI3K and oncogenic fusions including ALK, ROS1, and RET. The drugs can have a direct inhibitory effect such as EGFR inhibitors or indirect effects such as MEK1/2 and PI3 kinase inhibitors on KRAS and BRAF oncogenes.

Altogether the results point to a superior strategy by combining the cited drugs or other drugs combined with COT inhibitors as opposed to monotherapy of these drugs.

Sequence ID NO: 1
BAA03387.1 or NP_005195.2 or BAG36102.1 or CAG47079.1

| | |
|---|---|
| LOCUS | HUMPOPSTK 2763 bp mRNA linear PRI 16-FEB-2008 |
| DEFINITION | Homo sapiens mRNA for proto-oncogene protein, complete cds. |
| ACCESSION | D14497 |
| VERSION | D14497.1 |
| KEYWORDS | serine/threonine kinase. |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Euarchontoglires; Primates; Haplorrhini; |
| | Catarrhini; Hominidae; Homo. |
| REFERENCE 1 | |
| AUTHORS | Miyoshi, J., Higashi,T., Mukai, H., Ohuchi, T. and Kakunaga, T. |
| TITLE | Structure and transforming potential of the human cot oncogene |
| | encoding a putative protein kinase |
| JOURNAL | Mol. Cell. Biol. 11(8), 4088-4096 (1991) |
| PUBMED | 2072910 |
| REFERENCE | 2 (bases 1 to 2763) |
| AUTHORS | Aolci, M., Sugimoto, T., Sumida, S., Hamada, F., Alciyama, T. and |
| | Toyoshima, K. |
| TITLE | The human cot proto-oncogene encodes two protein serine/threonine |
| | kinases with different transforming activities by alternative |
| | initiation of translation |
| JOURNAL | Unpublished |
| REFERENCE | 3 (bases 1 to 2763) |
| AUTHORS | Aolci, M. |
| TITLE | Direct Submission |
| JOURNAL | Submitted (23-FEB-1993) Contact: Masahiro Aoki Res. Inst. for |
| | Microbiol Deseases, Osaka University, Department of Virology; 3-1 |
| | Yamadaoka, Suita, Osaka 565, Japan |
| COMMENT | On Feb 9, 1994 this sequence version replaced gi:433203. |
| FEATURES | Location/Qualifiers |
| source | 1..2763 |
| | /organism = ″Homo sapiens″ |
| | /mol_type = ″mRNA″ |
| | /db_xref = ″taxon:9606″ |
| | /cell_line = ″PLC/PRF/5″ |
| | /tissue_type = ″hepatoma″ |
| CDS | 367..1770 |
| | /function = ″serine/threonine kinase″ |
| | /codon_start = 1 |
| | /product = ″proto-oncogene protein″ |
| | /protein_id = ″BAA03387.1″ |

/translation = ″MEYMSTGSDNKEEIDLLIKHLNVSDVIDIMENLYASEEPAVYEPSLMTMCQDSNQND
ERSKSLLLSGQEVPWLSSVRYGTVEDLLAFANHISNTAKHEYGQRPQESGILLNMVITPQNGRYQI
DSDVLLIPWKLTYRNIGSDFIPRGAFGKVYLAQDIKTKKRMACKLIPVDQFKPSDVEIQACFRHENI
AELYGAVLWGETVHLFMEAGEGGSVLEKLESCGPMREFEIIWVTKHVLKGLDFLHSKKVIHHDIK
PSNIVEMSTKAVLVDEGLSVQMTEDVYFPKDLRGTEIYMSPEVILCRGHSTKADIYSLGATLIHMQT
GTPPWVKRYPRSAYPSYLYIIHKQAPPLEDIADDCSPGMRELIEASLERNPNHRPRAADLLKHEALN
PPREDQPRCTSLDSALLERKRLLSRKELELPENIADSSCTGSTEESEMLKRQRSLYIDLGALAGYFNL
VRGPPTLEYG″
SEQ ID NO: 2

| | |
|---|---|
| misc_feature | 1558 |
| | /note = ″rearranged point″ |
| | /citation = [1] |
| regulatory | 2696..2701 |
| | /regulatory_class = ″polyA_signal_sequence″ |
| regulatory | 2742..2747 |
| | /regulatory_class = ″polyA_signal_sequence″ |
| polyA_site | 2763 |
| ORIGIN | |
| 1 | ggatcccagt ggcccggcgt gctcggctcc cacaggcctg cagccagcat cgcaccgaac |
| 61 | cttcgggggg ccgcggctgg agcgctcggc cggcgtggga gcgcaaggcc gcagatgcaa |
| 121 | tcttcttacc gcgaagaagc caggggaata ggtagccaca tcttgtttgc agataagaaa |
| 181 | ggaagctaac gcagtatctg caaagccagg agtctgactc agtacttttc tcactcatgc |
| 241 | atacaagcag ctaaaaatga cacagcttat ttaccatgcc cctgacactg cactgagcac |
| 301 | tttatgagct tgaactctgt taatctcacg accacctcat gagactctcc agaaagagca |
| 361 | acagtaatgg agtacatgag cactggaagt gacaataaag aagagattga tttattaatt |
| 421 | aaacatttaa atgtgtctga tgtaatagac attatggaaa atctttatgc aagtgaagag |
| 481 | ccagcagttt atgaacccag tctaatgacc atgtgtcaag acagtaatca aaacgatgag |
| 541 | cgttctaagt ctctgctgct tagtggccaa gaggtaccat ggttgtcatc agtcagatat |
| 601 | ggaactgtgg aggatttgct tgcctttgca aaccatatat ccaacactgc aaagcatttt |
| 661 | tatggacaac gaccacagga atctggaatt ttattaaaca tggtcatcac tccccaaaat |
| 721 | ggacgttacc aaatagattc cgatgttctc ctgatcccct ggaagctgac ttacaggaat |
| 781 | attggttctg attttattcc tcggggcgcc tttggaaagg tatacttggc tcaagatata |
| 841 | aagacgaaga aaagaatggc gtgtaaactg atcccagtag atcaatttaa gccatctgat |
| 901 | gtggaaattc aggcttgctt ccggcacgag aacatcgcag agctgtatgg cgcagtcctg |
| 961 | tggggtgaaa ctgtccatct ctttatggaa gcaggcgagg gagggtctgt tctggagaaa |
| 1021 | ctggagagct gtggaccaat gagagaattt gaaattattt gggtgacaaa gcatgttctc |
| 1081 | aagggacttg attttctaca ctcaaagaaa gtgatccatc atgatattaa acctagcaac |
| 1141 | attgttttca tgtccacaaa agctgttttg gtggattttg gcctaagtgt tcaaatgacc |
| 1201 | gaagatgtct attttcctaa ggacctccga ggaacagaga tttacatgag cccagaggtc |
| 1261 | atcctgtgca ggggccattc aaccaaagca gacatctaca gcctgggggc cacgctcatc |
| 1321 | cacatgcaga cgggcacccc accctgggtg aagcgctacc ctcgctcagc ctatccctcc |
| 1381 | tacctgtaca taatccacaa gcaagcacct ccactggaag acattgcaga tgactgcagt |
| 1441 | ccagggatga gagagctgat agaagcttcc ctggagagaa accccaatca ccgcccaaga |
| 1501 | gccgcagacc tactaaaaca tgaggccctg aacccgccca gagaggatca gccacgctgt |
| 1561 | acgagtctgg actctgccct cttggagcgc aagaggctgc tgagtaggaa ggagctggaa |
| 1621 | cttcctgaga acattgctga ttcttcgtgc acaggaagca ccgaggaatc tgagatgctc |
| 1681 | aagaggcaac gctctctcta catcgacctc ggcgctctgg ctggctactt caatcttgtt |
| 1741 | cggggaccac caacgcttga atatggctga aggatgccat gtttgcctct aaattaagac |
| 1801 | agcattgatc tcctggaggc tggttctgct gcctctacac aggggcccgt tacagtgaat |
| 1861 | ggtgccattt tcgaaggagc agtgtgacct cctgtgaccc atgaatgtgc ctccaagcgg |
| 1921 | ccctgtgtgt ttgacatgtg aagctatttg atatgcacca ggtctcaagg ttctcatttc |
| 1981 | tcaggtgacg tgattctaag gcaggaattt gagagttcac agaaggatcg tgtctgctga |
| 2041 | ctgtttcatt cactgtgcac tttgctcaaa attttaaaaa taccaatcac aaggataata |
| 2101 | gagtagccta aaattactat tcttggttct tatttaagta tggaatattc attttactca |
| 2161 | gaatagcctg ttttgtgtat attggtgtat attatataac tctttgagcc tttattggta |
| 2221 | aattctggta tacattgaat tcattataat ttgggtgact agaacaactt gaagattgta |
| 2281 | gcaataagct ggactagtgt cctaaaaatg gctaactgat gaattagaag ccatctgaca |
| 2341 | gacggccact agtgacagtt tcttttgtgt tcctatggaa acattttata ctgtacatgc |
| 2401 | tatgctgaag acattcaaaa cgtgatgttt tgaatgtgga taaaactgtg taaaccacat |
| 2461 | aattttgtac atccaaggat gaggtgtgac ctttaagaaa aatgaaaact tttgtaaatt |
| 2521 | attgatgatt ttgtaattct tatgactaaa ttttctttta agcatttgta tattaaaata |
| 2581 | gcatactgtg tatgttttat atcaaatgcc ttcatgaatc tttcatacat atatatattt |
| 2641 | gtaacatgta aagtatgtga gtagtcttat gtaaagtatg tttttacatt atgcaaataa |
| 2701 | aacccaatac ttttgtccaa tgtggttggt caaatcaact gaataaattc agtattttgc |
| 2761 | ctt |

MAP3K8 mitogen-activated protein kinase kinase kinase 8 [Homo sapiens (human)]
Gene ID: 1326, updated on 31 Jan. 2019

### Summary

Official Symbol: MAP3K8 provided by HGNC
Official Full Name: mitogen-activated protein kinase kinase kinase 8 provided by HGNC
Primary source: HGNC:HGNC:6860
See, related: Ensembl:ENSG00000107968 MIM:191195
Gene type: protein coding
RefSeq status: REVIEWED
Organism: Homo sapiens

### Lineage: Eukaryota; Metazoa; Chordata; Craniata; Vertebrata; Euteleostomi; Mammalia; Eutheria; Euarchontoglires; Primates; Haplorrhini; Catarrhini; Hominidae; Homo

Also known as: COT; EST; ESTF; TPL2; AURA2; MEKK8; Tpl-2; c-COT

### Summary

This gene is an oncogene that encodes a member of the serine/threonine protein kinase family. The encoded protein localizes to the cytoplasm and can activate both the MAP kinase and JNK kinase pathways. This protein was shown to activate IkappaB kinases, and thus induce the nuclear production of NF-kappaB. This protein was also found to promote the production of TNF-alpha and IL-2 during T lymphocyte activation. This gene may also utilize a downstream in-frame translation start codon, and thus produce an isoform containing a shorter N-terminus. The shorter isoform has been shown to display weaker transforming activity. Alternate splicing results in multiple transcript variants that encode the same protein. [provided by RefSeq, September 2011]

What is claimed is:

1. A method of treating a patient suffering from a MAP3K8 (COT) positive cancer by administration of an effective amount of a combination therapy comprising a COT inhibitor and at least one therapeutic agent selected from consisting of: (i) growth factor inhibitors and growth factor receptor inhibitors; (ii) Fusion proto-oncogene inhibitors; (iii) proto-oncogene GTPases of 19 to 23 kDa and associated proteins inhibitors; (iv) proto-oncogenic cytoplasmic tyrosine and serine/threonine kinases inhibitors; (v) multi-kinase inhibitors and (vi) cell cycle or DNA repair inhibitors.

2. The method of claim 1 wherein the cancer therapeutic is selected from the group of a growth factor inhibitor and growth factor receptor inhibitor.

3. The method of claim 2 wherein the cancer therapeutic is selected from the group consisting of Osimertinib, Afatinib, Panitumumab, Cetuximab, Trastuzumab, Crizotinib, and Imatinib.

4. The method of claim 1 wherein the cancer therapeutic is a fusion proto-oncogene inhibitor.

5. The method of claim 4 wherein the cancer therapeutic is selected from the group consisting of Alectinib, Crizotinib, Ceritinib, Brigatinib and Lorlatinib.

6. The method of claim 1 wherein the cancer therapeutic is a cell cycle or DNA repair inhibitor.

7. The method of claim 6 wherein the cancer therapeutic is selected from the group consisting of Abemaciclib, Trilaciclib, niraparib, olaparib, rucaparib, and talazoparib.

8. The method of claim 1 wherein the cancer therapeutic is a proto-oncogene GTPase of 19 to 23 kDa and associated proteins.

9. The method of claim 8 wherein the cancer therapeutic is selected from the group consisting of (but not limited to) ARS-853; ARS-1620; AMG-510; MTRX849 (HRAS, NRAS and KRAS inhibitors), tipifarnib, lonafarnib, bms-214662, 1778123 (farnesyl transferase inhibitors), deltarasin (KRAS-PDEδ inhibitors), sulindac-derived compounds (Ras-Raf interaction inhibitors), and Kobe0065 and Kobe2602 (SOS binding inhibitors).

10. The method of claim 1 wherein the cancer therapeutic is a protooncogenic cytoplasmic tyrosine kinase, serine/threonine kinase or membrane lipid kinase.

11. The method of claim 10 wherein the cancer therapeutic is selected from the group consisting of: Trametinib, Binimetinib and Sorafenib (MEK1/2 inhibitors, (ii) SCH772984, GDC-0994, Ulixertinib, and LY3214996 (ERK1/2 inhibitors), (iii) Duvelisib, Copanlisib, and Copanlisib (PI3K inhibitors), (iv) Everolimus, Sirolimus and Temsirolimus (mTOR inhibitors), and (v) idelalisib (AKT inhibitors).

12. The method of claim 1 wherein the cancer therapeutic is a multi-kinase inhibitor.

13. The method of claim 12 wherein the cancer therapeutic is selected from the group consisting of neratinib, ponatinib, regorafenib, sorafenib, cabozantinib, lenvatinib, vandetanib.

14. The method of claim 1 wherein the MAP3K8 (COT) positive cancer is selected from the group consisting of: pancreatic cancer, renal cancer, breast cancer, bladder cancer, leukemia, acute myeloid leukemia, thyroid cancer, colorectal cancer, prostate cancer, uterine carcinosarcoma, uterine cancer, bladder urothelial carcinoma, uterine corpus endometrial carcinoma, gastric adenocarcinoma, cervical adenocarcinoma, hepatocellular cancer, lung cancer (NSCLC, SCLC, lung adenocarcinoma, lung squamous cell carcinoma), glioblastoma multiforme, glioblastoma, brain cancer, ovarian cancer, cervical cancer, gastric cancer, esophageal cancer, head and neck cancer, melanoma, skin cancer, neuroendocrine cancers, multiple myeloma, brain tumors (e.g., adult glioblastoma multiforme; glioma, anaplastic oligodendroglioma, and adult anaplastic astrocytoma), child brain tumor, bone cancer, sarcoma, CNS cancer, ovarian cancer, renal cancer, prostate cancer, or breast cancer.

15. A method of evaluating a patient with cancer selected from the group consisting of: where the cancer has an oncogene status of either elevated expression levels of an oncogene or harboring an oncogenic mutation where the oncogene is selected from the group consisting: (i) growth factor and growth factor receptor oncogene; (ii) Fusion of proto-oncogenes; (iii) GTPases oncogene of 19 to 23 Kda and/or associated proteins as supporting oncogenic activity; and, (iv) oncogenic cytoplasmic tyrosine and serine/threonine kinases. where the method comprises the following steps:
(i) determining the MAP3K8 (COT) status in patient tumor cells from a patient sample;
(ii) comparing the levels of COT expression or activation (by phosphorylation) from step (a) to a threshold activity level of COT derived from a cohort of cells from at least 200 test individuals where the cells have a defined level of COT activation/expression that is either negative expression or positive expression where the cohort of cells represent a cancer having a positive oncogene status;
(iii) determining the oncogene status of the cancer cells from the patient; and,
(iv) identifying the patient as potentially responding therapeutically to a combination of a COT inhibitor and an oncogene inhibitor that is known to therapeutically treat cancers matching the oncogene status of step iii.

16. A method of claim 15 wherein the cancer is a non-small-cell lung cancer.

17. The method of claim 15 where the oncogene status relates to one of the oncogenes selected from the group consisting of (i) growth factor and growth factor receptor inhibitors; (ii) Fusion proto-oncogene inhibitors; (iii) proto-oncogene GTPases of 15 to 20 Kda and associated proteins; and, (iv) proto-oncogenic cytoplasmic tyrosine and serine/threonine kinases; (v) multi-kinase inhibitors; and (vi) cell cycle or DNA repair inhibitors.

18. The method of claim 15 wherein the COT status is determined by amplification of mRNA encoding COT or the phosphorylation of COT or copy number of COT or mutation of COT or COT overexpression (elevated protein level).

19. The method of claim 15 wherein the COT status is determined on patient tumor (biopsy or liquid biopsy) utilizing a method selected from the group consisting of polymerase chain reaction, isothermal amplification (PCR), Immuno-histochemistry with or without anti phopho antibodies to Thr290 of COT, Next Generation Sequencing, liquid biopsy, and direct biopsy.

20. A method of treating a patient hosting a MAP3K8 positive cancer, the method comprising:
i. selecting a patient with a cancer selected from the group consisting of: where the cancer has an oncogene status of either elevated expression levels of an oncogene or harboring an oncogenic mutation where the oncogene is selected from the group consisting: (i) growth factor and growth factor receptor oncogene; (ii) Fusion of proto-oncogenes; (iii) GTPases oncogene of 19 to 23 Kda and/or associated proteins as supporting oncogenic activity; and, (iv) oncogenic cytoplasmic tyrosine and serine/threonine kinases; where the patient is also determined to have cancer cells having an elevated MAP3K8 (COT) status where that elevated COT status is determined by comparing the levels of COT expression or activation (by phosphorylation) from step to a threshold activity level of COT derived from a cohort of cells from at least 200 test individuals where the cells have a defined level of COT activation/expression that is either negative expression or positive expression where the cohort of cells represent a cancer having a positive oncogene status
and,
(ii) treating the patient with a therapeutically effective amount of a combination of a COT inhibitor and an oncogene inhibitor that is known to therapeutically treat cancers matching the oncogene status of step i.
